(19) United States
(12) Patent Application Publication
(10) Pub. No.: US 2012/0177605 A1
(43) Pub. Date: Jul. 12, 2012

(54) Delivery of Polynucleotides Across the Blood-Brain-Barrier Using Recombinant AAV9

(75) Inventors: Brian K. Kaspar, Westerville, OH (US); Kevin Foust, Westerville, OH (US)

(73) Assignee: Nationwide Children's Hospital Inc., Columbus, OH (US)

(21) Appl. No.: 13/270,840

(22) Filed: Oct. 11, 2011

Related U.S. Application Data
(63) Continuation of application No. 13/035,777, filed on Feb. 25, 2011, now abandoned.
Application No. 13/035,777 is a continuation-in-part of application No. PCT/US2009/068818, filed on Dec. 18, 2009.
(60) Provisional application No. 61/139,470, filed on Dec. 19, 2008.
(60) Provisional application No. 61/308,884, filed on Feb. 26, 2010.

(51) Int. Cl. A61K 48/00; A61P 21/00; A61P 25/28; C12N 15/63; A61P 25/00

(52) U.S. Cl. 424/93.2; 435/320.1

(57) ABSTRACT

The present invention relates to methods and materials useful for systemically delivering polynucleotides to the spinal cord. Use of the methods and materials is indicated, for example, for treatment of lower motor neuron diseases such as spinal muscular atrophy (SMA) and amyotrophic lateral sclerosis (ALS) as well as Pompe disease and lysosomal storage disorders.

### PRIORITY CLAIM

The present application claims the benefit of priority of U.S. Provisional Application No. 61/308,884, filed Feb. 26, 2010, and is also a continuation-in-part of International Patent Application No. PCT/US09/68818, filed Dec. 18, 2009, which claims the benefit of priority of U.S. Provisional Application 61/139,470, filed Dec. 19, 2008.

### STATEMENT OF GOVERNMENT INTEREST

This invention was made with Government support under R21EY018491 awarded by the National Institutes of Health (NIH)/National Eye Institute (NEI), and under R21NS064328, awarded by the NIH/National Institute of Neurological Disorders and Stroke (NINDS). The Government has certain rights in the invention.

### FIELD OF THE INVENTION

The present invention relates to Adeno-associated virus 9 methods and materials useful for systemically delivering polynucleotides across the blood brain barrier. Accordingly, the present invention also relates to methods and materials useful for systemically delivering polynucleotides to the central and peripheral nervous systems. Use of the methods and materials is indicated, for example, for treatment of lower motor neuron diseases such as spinal muscular atrophy and amyotrophic lateral sclerosis as well as Pompe disease and lysosomal storage disorders.

### BACKGROUND

Large-molecule drugs do not cross the blood-brain-barrier (BBB) and 98% of small-molecules cannot penetrate this barrier, thereby limiting drug development efforts for many CNS disorders [Pardridge, W. M. Nat Rev Drug Discov 1: 131-139 (2002)]. Gene delivery has recently been proposed as a method to bypass the BBB [Kaspar, et al., Science 301: 839-842 (2003)]; however, widespread delivery to the brain and spinal cord has been challenging. The development of successful gene therapies for motor neuron disease will likely require widespread transduction within the spinal cord and motor cortex. Two of the most common motor neuron diseases are spinal muscular atrophy (SMA) and amyotrophic lateral sclerosis (ALS), both debilitating disorders of children and adults, respectively, with no effective therapies to date. Recent work in rodent models of SMA and ALS involves gene delivery using viruses that are retrogradely transported following intramuscular injection [Kaspar et al., Science 301: 839-842 (2003); Azzouz et al., J Clin Invest 114: 1726-1731 (2004); Azzouz et al., Nature 429: 413-417 (2004); Ralph et al. Nat Med 11: 429-433 (2005)]. However, clinical development may be difficult given the numerous injections required to target the widespread region of neurodegeneration throughout the spinal cord, brainstem and motor cortex to effectively treat these diseases. AAV vectors have also been used in a number of recent clinical trials for neurological disorders, demonstrating sustained transgene expression, a relatively safe profile, and promising functional responses, yet have required surgical intraparenchymal injections [Kaplitt et al., Lancet 369: 2097-2105 (2007); Marks et al., Lancet Neurol 7: 400-408 (2008); Worgall et al., Hum Gene Ther (2008)].

SMA is an early pediatric neurodegenerative disorder characterized by flaccid paralysis within the first six months of life. In the most severe cases of the disease, paralysis leads to respiratory failure and death usually by two years of age. SMA is the second most common pediatric autosomal recessive disorder behind cystic fibrosis with an incidence of 1 in 6000 live births. SMA is a genetic disorder characterized by the loss of lower motor neurons (LMNs) residing along the length of the entire spinal cord. SMA is caused by a reduction in the expression of the survival motor neuron (SMN) protein that results in denervation of skeletal muscle and significant muscle atrophy. SMN is a ubiquitously expressed protein that functions in U snRNP biogenesis.

In humans there are two very similar copies of the SMN gene termed SMN1 and SMN2. The amino acid sequence encoded by the two genes is identical. However, there is a single, silent nucleotide change in SMN2 in exon 7 that results in exon 7 being excluded in 80-90% of transcripts from SMN2. The resulting truncated protein, called SMNΔ7, is less stable and rapidly degraded. The remaining 10-20% of transcript from SMN2 encodes the full length SMN protein. Disease results when all copies of SMN1 are lost, leaving only SMN2 to generate full length SMN protein. Accordingly, SMN2 acts as a phenotypic modifier in SMA in that patients with a higher SMN2 copy number generally exhibit later onset and less severe disease.

To date, there are no effective therapies for SMA. Therapeutic approaches have mainly focused on developing drugs for increasing SMN levels or enhancing residual SMN function. Despite years of screening, no drugs have been fully effective for increasing SMN levels as a restorative therapy. A number of mouse models have been developed for SMA. See, Hsieh-Li et al., Nature Genetics, 24 (1): 66-70 (2000); Le di al., Hum. Mol. Genet., 14 (6): 845-857 (2005); Monani et al., J. Cell. Biol., 160 (1): 41-52 (2003) and Monani et al., Hum. Mol. Genet., 9 (3): 333-339 (2000). A recent study express a full length SMN cDNA in a mouse model and the authors concluded that expression of SMN in neurons can have a significant impact on symptoms of SMA. See Gavrilina et al., Hum. Mol. Genet., 17 (8):1063-1075 (2008).

ALS is another disease that results in loss of muscle and/or muscle function. First characterized by Charcot in 1869, it is a prevalent, adult-onset neurodegenerative disease affecting nearly 5 out of 100,000 individuals. ALS occurs when specific nerve cells in the brain and spinal cord that control voluntary movement gradually degenerate. Within two to five years after clinical onset, the loss of these motor neurons leads to progressive atrophy of skeletal muscles, which results in loss of muscular function resulting in paralysis, speech deficits, and death due to respiratory failure.

The genetic defects that cause or predispose ALS onset are unknown, although missense mutations in the SOD-1 gene occurs in approximately 10% of familial ALS cases, of which up to 20% have mutations in the gene encoding Cu/Zn superoxide dismutase (SOD1), located on chromosome 21. SOD-1 normally functions in the regulation of oxidative stress by conversion of free radical superoxide anions to hydrogen peroxide and molecular oxygen. To date, over 90 mutations have been identified spanning all exons of the SOD-1 gene. Some of these mutations have been used to generate lines of transgenic mice expressing mutant human SOD-1 to model the progressive motor neuron disease and pathogenesis of ALS.

SMA and ALS are two of the most common motor neuron diseases. Recent work in rodent models of SMA and ALS has examined treatment by gene delivery using viruses that are retrogradedly transported following intramuscular injection. See Azzouz et al., J. Clin. Invest., 114: 1726-1731 (2004); Kaspar et al., Science, 301: 839-842 (2003); Azzouz et al., Nature, 429: 413-417 (2004) and Ralph et al., Nature Medicine, 11: 429-433 (2005). Clinical use of such treatments may be difficult given the numerous injections required to target neurodegeneration throughout the spinal cord, brainstem and motor cortex.

Adeno-associated virus (AAV) is a replication-deficient parvovirus, the single-stranded DNA genome of which is about 4.7 kb in length including 145 nucleotide inverted terminal repeat (ITRs). The nucleotide sequence of the AAV serotype 2 (AAV2) genome is presented in Srivastava et al., J Virol, 45: 555-564 (1983) as corrected by Ruffing et al., J Gen Viral, 75: 3385-3392 (1994). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple serotypes of AAV exist and offer varied tissue tropism. Known serotypes include, for example, AAV1, AAV2, AAV3, AAV4, AAV5, AAV6, AAV7, AAV8, AAV9, AAV10 and AAV11. AAV9 is described in U.S. Pat. No. 7,198,951 and in Gao et al., J. Virol., 78: 6381-6388 (2004). Advances in the delivery of AAV6 and AAV8 have made possible the transduction by these serotypes of skeletal and cardiac muscle following simple systemic intravenous or intraperitoneal injections. See Pacak et al., Circ. Res., 99 (4): 3-9 (1006) and Wang et al., Nature Biotech., 23 (3): 321-8 (2005). The use of AAV to target cell types within the central nervous system, though, has required surgical intraparenchymal injection. See, Kaplitt et al., supra; Marks et al., supra and Worgall et al., supra.

There thus remains a need in the art for methods and vectors for delivering genes across the BBB.

### SUMMARY

The present invention provides methods and materials useful for systemically delivering polynucleotides across the BBB.

In one embodiment, the invention provides a method of delivering a polynucleotide across the BBB comprises systemically administering a rAAV9 with a genome including the polynucleotide to a patient. In some embodiments the rAAV9 genome is a self complementary genome. In other embodiments the rAAV9 genome is a single-stranded genome.

The present invention also provides methods and materials useful for systemically delivering polynucleotides across the blood brain barrier to the central and peripheral nervous system. Accordingly in another embodiment, a method is provided of delivering a polynucleotide to the central nervous system comprising systemically administering a rAAV9 with a self-complementary genome including the genome to a patient. In another embodiment, a method of delivering a polynucleotide to the peripheral nervous system comprising systemically administering a rAAV9 with a self-complementary genome including the polynucleotide to a patient is provided.

In some embodiments, the polynucleotide is delivered to brain. In other embodiments, the polynucleotide is delivered to the spinal cord. In still other embodiments, the polynucleotide is delivered to a lower motor neuron. Embodiments of the invention employ rAAV9 to deliver polynucleotides to nerve and glial cells. In some aspects, the glial cull is a microglial cell, an oligodendrocyte or an astrocyte. In other aspects the rAAV9 is used to deliver a polynucleotide to a Schwann cell.

The development of the rat brain has been characterized as including four stages [McIlwain. Chemical and enzymic make-up of the brain during development: In: Biochemistry and the Central Nervous System. Churchill, London. 270-299 (1966)]. Stage one includes the fetal period during which cell division produces 94-97% of the number of cells found in the adult brain. Stage two extends from birth, when the brain is 15% of the adult weight, to ten days postnatal at which time the major growth in size has been produced by the growth of cells, especially axons and dendrites. During the third stage, from 10-20 days, when the rate of growth is much reduced, new processes such as myelinization and electrical activity first occur. The fourth stage, beyond 20 days, is occupied by slow overall growth. Tight junctions between cerebral endothelial cells are functional in the developing brain, whereas the intimate associations of astrocytic endfeet are not complete until about 3 weeks of age [Caley et al., J. Comp. Neurol. 138: 31-47 (1970)]. Further, early in development, the immature blood vessels are contiguous with extracellular spaces, cell bodies, and an assortment of cell processes including astrocytic end-feet. As the tissue matures the vessels become increasingly covered by the astrocyte end-feet with concomitant diminution of the surrounding extracellular spaces. By nine days, most of the capillaries are almost completely covered by astrocyte end-feet, and the extracellular spaces are reduced but not entirely gone. At 21 days, the large extracellular spaces are gone, and the capillary is completely covered by contiguous astrocyte end-feet joined to each other [Caley et al., J. Comp. Neurol. 138: 31-47 (1970)].

In humans, it is thought that the permeability of the BBB is inure than that of an adult for up to 6 months after birth [Watson et al., Birth Defects Research (Part 13) 77: 471-484 (2006)]. An example of this can be seen in the toxicity profile of methylmercury. In human adults, methylmercury exposure causes damage in specific areas, such as the granule cell layer of the cerebellum and the visual cortex of the cerebrum, but in babies exposed in utero or at an early postnatal age, the damage is more extensive. A potential reason for this is incomplete development of the BBB [Costa et al., Ann Rev Pharmacol Toxicol 44: 87-110 (2004)].

The development of the BBB in humans is a gradual process, beginning in utero and acquiring capabilities similar to that of an adult at approximately 6 months of age [Costa et al., Ann Rev Pharmacol Toxicol 44: 87-110 (2004)]. It is generally believed that development of the BBB begins shortly after intraneural neovascularization [Bauer et al., Cell Mol Neurobiol 20: 13-28 (2000)]. The formation of tight junction associated transmembrane proteins, occludin and claudin-5, both involved in BBB function, occurs during gestation [Virgintino et al., Histochem Cell Biol 122: 51-59 (2004)]. At 14 weeks of gestation, the immunosignal for these proteins shifts from presence in the cytoplasm prior to this point, to the interface of endothelial cells, forming a linear pattern of immunoreactivity where one would expect the BBB to be present [Virgintino et al., Histochem Cell Biol 122: 51-59 (2004)]. It is thought that structural and functional aspects of the BBB are similar in various species [Cserr et al., Am J Physiol 246: 277-287 (1984)].

Using injections of very high concentrations of trypan blue (enough to kill ⅓ of the animals), Behnsen [Zeit Zellforsch Mikrosk Anat 4: 515-572 (1905)] found that more dye was incorporated into the brains of mice up to 4 weeks of age compared to mice aged 5-8 weeks of age, suggesting that the early BBB is more permeable than that of the adult. Penta [Riv Neurol 5: 62-80 (1932)] reported that daily injections of high concentrations of trypan blue for 10-20 days postnatal led to some staining of brain tissue in the guinea pig and rat. Later studies supported this notion that the postnatal BBB was not particularly functional. Stewart et al. [Brain Res 429:271-281 (1987)] found that unfused endothelial cell outer “leaflets” in the BBB junction were more prevalent in fetuses than adults, and that there was a gradual decrease in unfused leaflets in postnatal animals. Vorbrodt et al. [Dev Neurosci 8: 1-13 (1986)] reported that mature expression of alkaline phosphatase in the BBB endothelial cells necessary for a fully functional 131313 appeared early in the postnatal mouse, from 12-24 days of age. In the rat, the permeability of the BBB is very high at birth and decreases in the first few weeks after birth [Clark et al., Dev Neurosci 15: 174-180 (1993)]. The uptake of amino acids into the brain decreases with increased age in both species, and this is often attributed to the development of the BBB, though it might also be due to variations in the efficiency of active transport systems [Ford, Prog Brain Res 40:1-12 (1973)]. Al-Sarraf et al. [Brain Res Dev Brain Res 102: 127-134 (1997)] reported maximal transport (Vmax) of the acidic amino acids aspartate and glutamate was 50% lower in 7-10-week-old rats compared to 1-week-old rats.

Other studies have indicated that the BBB is at least somewhat functional at an earlier timepoint. Using a lower concentration of trypan blue, in injections of the dye into rabbits, cats, mice, and rats on the day of birth or within a few days after birth, the dye did not penetrate into the brain [Stern et al., Compt Rendus Se'ances Soc Biol 96: 1149-1152 (1927)]. In the chick, the BBB gradually becomes impermeable to macromolecules at embryonic days 13-14, based on permeability to horseradish peroxidase. Similarly, the mouse BBB is impermeable to macromolecules before birth [Risau et al., Dev Biol 117: 537-545 (1986)].

As discussed in Abbott et al. [Nat Rev Neurosci 7: 41-53 (2006)], the BBB is a selective barrier formed by the endothelial cells that line cerebral microvessels [Risau et al., Trends Neurosci. 13: 174-178 (1990); Abbott et al., Mol. Med. Today 2: 106-113 (1996); Abbott, J. Anat. 200: 629-638 (2002); Begley et al., Prog. Drug Res. 61: 40-78 (2003)]. As discussed herein, the establishment of the BBB requires specialized endothelial tight junction cells, particular patterns of enzymatic activity, a distinct electrochemical gradient, and specific BBB transporters. The BBB acts as a ‘physical barrier’ because complex tight junctions between adjacent endothelial cells force most molecular traffic to take a transcellular route across the BBB, rather than moving paracellularly through the junctions, as in most endothelia [Wolburg et al., Vasc. Pharmacol. 38: 323-337 (2002); Hawkins et al., Pharmacol. Rev. 57: 173-185 (2005)]. The brain endothelium has a much lower degree of endocytosis/transcytosis activity than does peripheral endothelium, which contributes to the transport-barrier property of the BBB. Hence, the term ‘blood-brain barrier’ covers a range of passive and active features of the brain endothelium. As the tight junctions severely restrict entry of hydrophilic drugs, and there is limited penetration of larger molecules such as peptides, strategies for drug delivery to the CNS need to take these features into account.

The earliest histological studies have shown that brain capillaries are surrounded by or closely associated with several cell types, including the perivascular endfeet of astrocytic glia, pericytes, microglia and neuronal processes. In the larger vessels (arterioles, arteries and veins), smooth muscle forms a continuous layer, replacing pericytes [Iadecola, Nature Rev. Neurosci. 5: 347-360 (2004)]. Neuronal cell bodies are typically no more than ˜10 m from the nearest capillary [Schlageter et al., Microvasc. Res. 58: 312-328 (1999)]. These close cell-cell associations, particularly of astrocytes and brain capillaries, led to the suggestion that they could mediate the induction of the specific features of the barrier phenotype in the capillary endothelium of the brain [Davson et al., Proc. R. Soc. Med. 60: 326-328 (1967)].

Astrocytes show a number of different morphologies, depending on their location and association with other cell types. Of the ˜11 distinct phenotypes that can be readily distinguished, 8 involve specific interactions with blood vessels [Reichenbach et al. in Neuroglia 2nd edn (eds Kettemann, H. & Ransom, B. R.) 19-35 (Oxford Univ. Press, New York, 2004)]. There is strong evidence, particularly from studies in cell culture, that astrocytes can upregulate many BBB features, leading to tighter tight junctions (physical barrier) [Dehouck et al., J. Neurochem. 54: 1798-1801 (1990); Rubin et al., J. Cell Biol. 115: 1725-1735 (1991)], the expression and polarized localization of transporters, including Pgp24 and GLUT1 [McAllister et al., Brain Res. 409: 20-30 (2001)] (transport barrier), and specialized enzyme systems (metabolic barrier) [Abbott, J. Anat. 200: 629-638 (2002); Hayashi et al., Glia 19: 13-26 (1997); Sobue et al., Neurosci. Res. 35: 155-164 (1999); Haseloff et al., Cell. Mol. Neurobiol. 25: 25-39 (2005)]. Astrocytes are derived from ependymoglia of the developing neural tube, and retain some features of their original apical-basal polarity, together with more specific polarization of function in relation to particular cell-cell associations of the adult [Abbott, in Blood-Brain Interfaces—From Ontology to Artificial Barriers (eds Dermietzel, R., Spray, D. & Nedergaard, M.) 189-208 (Wiley-VCH, Weinheim, Germany, 2006); Reichenbach, A. & Wolburg, H. in Neuroglia 2nd edn (eds Kettemann, H. & Ransom, B. R.) 19-35 (Oxford Univ. Press, New York, 2004)]. The perivascular endfeet of astrocytes, which are closely applied to the microvessel wall, show several specialized features characteristic of this location, including a high density of orthogonal arrays of particles (OAPs) containing the water channel aquaporin 4 (AQP4) and the Kir4.1 K⁺ channel, which are involved in ion and volume regulation. The OAPs/AQP4 polarity of astrocytes correlates with the expression of agrin, a heparin sulphate proteoglycan, on the basal lamina [Wolburg et al., Vasc. Pharmacol. 38: 323-337 (2002); Verkman, J. Anat. 200: 617-627 (2002)]. Agrin accumulates in brain microvessels at the time of BBB tightening, and is important for the integrity of the BBB [Wolburg, H. in Blood-Brain Interfaces—from Ontogeny to Artificial Barriers (eds Dermietzel, R., Spray, D. & Nedergaard, M.) 77-107 (Wiley-VCH, Weinheim, Germany, 2006)].

Thus, in one embodiment the invention provides a method of delivering a polynucleotide across the BBB comprising systemically administering a rAAV9 with a genome including the polynucleotide to a patient, wherein the polynucleotide is administered to the patient prior to completion of formation of glial cell endfeet. In another embodiment, the invention provides a method of delivering a polynucleotide across the BBB comprising systemically administering a rAAV9 with a genome including the polynucleotide to a patient, wherein the polynucleotide is administered to the patient after completion of formation of glial cell endfeet.

In another embodiment, the invention provides a method of delivering a polynucleotide across the BBB comprising systemically administering a rAAV9 with a genome including the polynucleotide to a patient, wherein the rAAV9 is administered on postnatal day 1 (P1). In various aspects, the rAAV9 is administered on P2, P3, P4, P5, P6, P7, P8, P9, P10, P11, P12, P13, P14, P15, P16, P17, P18, P19, P20, P21, P22, P23, P24, P25, P26, P27, P28, P29, P30, P31, P32, P33, P34, P35, P36, P37, P38, P39, P40, P41, P42, P43, P44, P45, P46, P47, P48, P49, P50, P51, P52, P53, P54, P55, P56, 957. P58, P59, P60, P61, P62, P63, P64, P65, P66, P67, P68, P69, P70, P71, P72, P73, P74, P75, P76, P77, P78, P79, P80, P81, P82, P83, P84, P85, P86, P87, P88, P89, P90, P91, P92, P93, P94, P95, P96, P97, P98, P99, P100, P110, P120, P130, P140, P150, P160, P170, P180, P190, P200, P250, P300, P350, 1 year, 1.5 years, 2 years, 2.5 years, 3 years or older.

In another embodiment, a method of delivering a polynucleotide to vascular endothelial cells is provided comprising the step of systemically administering a rAAV9 comprising a self-complementary genome including the polynucleotide to a patient, wherein the polynucleotide is administered to the patient prior to completion of formation of glial cell endfeet. In a further embodiment, a method of delivering a polynucleotide across endothelial cell tight junctions of the blood brain barrier is provided comprising the step of systemically administering to a patient a rAAV9 comprising a self-complementary genome including the polynucleotide. In yet another embodiment, a method of delivering a polynucleotide to an astrocyte of the blood brain barrier is provided comprising the step of systemically administering to a patient a rAAV9 comprising a self-complementary genome including the polynucleotide. In various aspects of the embodiments, the polynucleotide is a SMN polynucleotide.

In those methods of the invention for systemically delivering polynucleotides to the spinal cord, use of the methods and materials is indicated, for example, for lower motor neuron diseases such as SMA and ALS as well as Pompe disease, lysosomal storage disorders, Glioblastoma multiforme and Parkinson's disease. Lysosomal storage disorders include, but are not limited to, Activator Deficiency/GM2 Gangliosidosis, Alpha-mannosidosis, Aspartylglucosaminuria, Cholesteryl ester storage disease, Chronic Hexosaminidase A Deficiency, Cystinosis, Danon disease, Fabry disease, Farber disease, Fucosidosis, Galactosialidosis, Gaucher Disease (Type I, Type II, Type III), GM1 gangliosidosis (Infantile, Late infantile/Juvenile, Adult/Chronic), I-Cell disease/Mucolipidosis II, Infantile Free Sialic Acid Storage Disease/ISSD, Juvenile Hexosaminidase A Deficiency, Krabbe disease (Infantile Onset, Late Onset), Metachromatic Leukodystrophy, Mucopolysaccharidoses disorders (Pseudo-Hurler polydystrophy/Mucolipidosis IIIA, MPSI Hurler Syndrome, MPSI Scheie Syndrome, MPS I Hurler-Scheie Syndrome, MPS II Hunter syndrome, Sanfilippo syndrome Type A/MPS III A, Sanfilippo syndrome Type B/MPS III B, Sanfilippo syndrome Type C/MPS III C, Sanfilippo syndrome Type D/MPS III D, Morquio Type A/MPS IVA, Morquio Type B/MPS IVB, MPS IX Hyaluronidase Deficiency, MPS VI Maroteaux-Lamy, MPS VII Sly Syndrome, Mucolipidosis I/Sialidosis, Mucolipidosis IIIC, Mucolipidosis type IV), Multiple sulfatase deficiency, Niemann-Pick Disease (Type A, Type B, Type C), Neuronal Ceroid Lipofuscinoses (CLN6 disease (Atypical Late Infantile, Late Onset variant, Early Juvenile), Batten-Spielmeyer-Vogt/Juvenile NCL/CLN3 disease, Finnish Variant Late Infantile CLN5, Jansky-Bielschowsky disease/Late infantile CLN2/TPP1 Disease, Kufs/Adult-onset NCL/CLN4 disease, Northern Epilepsy/variant late infantile CLN8, Santavuori-Haltia/Infantile CLN1/PPT disease, Beta-mannosidosis, Pompe disease/Glycogen storage disease type II, Pycnodysostosis, Sandhoff Disease/Adult Onset/GM2 Gangliosidosis, Sandhoff Disease/GM2 gangliosidosis—Infantile, Sandhoff Disease/GM2 gangliosidosis—Juvenile, Schindler disease, Salla disease/Sialic Acid Storage Disease, Tay-Sachs/GM2 gangliosidosis, Wolman disease.

In further embodiments, use of the methods and materials is indicated for treatment of nervous system disease such as Rett Syndrome, Alzheimer's Disease, Parkinson's Disease, Huntington's Disease along with nervous system injury including spinal cord and brain trauma/injury, stroke, and brain cancers.

In one aspect, the invention provides rAAV genomes. The rAAV genomes comprise one or more AAV ITRs flanking a polynucleotide encoding a polypeptide (including, but not limited to, an SMN polypeptide) or encoding short hairpin RNAs directed at mutated proteins or control sequences of their genes. The polynucleotide is operatively linked to transcriptional control DNAs, specifically promoter DNA and polyadenylation signal sequence DNA that are functional in target cells to form a gene cassette. The gene cassette may also include intron sequences to facilitate processing of an RNA transcript when expressed in mammalian cells.

In some aspects, the rAAV9 genome encodes atrophic or protective factor. In various embodiments, use of a trophic or protective factor is indicated for neurodegenerative disorders contemplated herein, including but not limited to Alzheimer's Disease, Parkinson's Disease, Huntington's Disease along with nervous system injury including spinal cord and brain trauma/injury, stroke, and brain cancers. Non-limiting examples of known nervous system growth factors include nerve growth factor (NGF), brain-derived neurotrophic factor (BDNF), neurotrophin-3 (NT-3), neurotrophin-4/5 (NT-4/5), neurotrophin-6 (NT-6), ciliary neurotrophic factor (CNTF), glial cell line-derived neurotrophic factor (GDNF), the fibroblast growth factor family (e.g., FGF's 1-15), leukemia inhibitory factor (LIF), certain members of the insulin-like growth factor family (e.g., IGF-1), the neurturins, persephin, the bone morphogenic proteins (BMPs), the immunophilins, the transforming growth factor (TGF) family of growth factors, the neuregulins, epidermal growth factor (EGF), platelet-derived growth factor (PDGF), vascular endothelial growth factor family (e.g. VEGF 165), follistatin, Hifl, and others. Also generally contemplated are zinc finger transcription factors that regulate each of the trophic or protective factors contemplated herein. In further embodiments, methods to modulate neuro-immune function are contemplated, including but not limited to, inhibition of microglial and astroglial activation through, for example, NFkB inhibition, or NFkB for neuroprotection (dual action of NFkB and associated pathways in different cell types.) by siRNA, shRNA, antisense, or miRNA. In still further embodiments, the rAAV9 genome encodes an apoptotic inhibitor (e.g., bcl2, bclxL). Use of a rAAV9 encoding a trophic factor or spinal cord injury modulating protein or a suppressor of an inhibitor of axonal growth (e.g., a suppressor of Nogo [Oertle et al., The Journal of Neuroscience, 23 (13):5393-5406 (2003)] is also contemplated for treating spinal cord injury.

In some embodiments, use of materials and methods of the invention is indicated for neurodegenerative disorders such as Parkinson's disease. In various embodiments, the rAAV9 genome may encode, for example, Aromatic acid dopa decarboxylase (AADC), Tyrosine hydroxylase, GTP-cyclohydrolase 1 (gtpch1), apoptotic inhibitors (e.g., bcl2, bclxL), glial cell line-derived neurotrophic factor (GDNF), the inhibitory neurotransmitter-amino butyric acid (GABA), and enzymes involved in dopamine biosynthesis. In further embodiments, the rAAV9 genome may encode, for example, modifiers of Parkin and/or synuclein.

In some embodiments, use of materials and methods of the invention is indicated for neurodegenerative disorders such as Alzheimer's disease. In further embodiments, methods to increase acetylcholine production are contemplated. In still further embodiments, methods of increasing the level of a choline acetyltransferase (ChAT) or inhibiting the activity of an acetylcholine esterase (AchE) are contemplated.

In some embodiments, the rAAV9 genome may encode, for example, methods to decrease mutant Huntington protein (htt) expression through siRNA, shRNA, antisense, and/or miRNA for treating a neurodegenerative disorder such as Huntington's disease.

In some embodiments, use of materials and methods of the invention is indicated for neurodegenerative disorders such as ALS. In some aspects, treatment with the embodiments contemplated by the invention results in a decrease in the expression of molecular markers of disease, such as TNFα, nitric oxide, peroxynitrite, and/or nitric oxide synthase (NOS).

In other aspects, the vectors could encode short hairpin RNAs directed at mutated proteins such as superoxide dismutase for ALS, or neurotrophic factors such as GDNF or IGF1 for ALS or Parkinson's disease.

In some embodiments, use of materials and methods of the invention is indicated for preventing or treating neurodevelopmental disorders such as Rett Syndrome. For embodiments relating to Rett Syndrome, the rAAV9 genome may encode, for example, methyl cytosine binding protein 2 (MeCP2).

In various embodiments, use of the materials and methods of the present disclosure results in amelioration of at least one symptom of a disease or disorder.

The rAAV genomes of the invention lack AAV rep and cap DNA. AAV DNA in the rAAV genomes (e.g., ITRs) may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10 and AAV-11. The nucleotide sequences of the genomes of the AAV serotypes are known in the art. For example, the complete genome of AAV-1 is provided in GenBank Accession No. NC_{—}002077; the complete genome of AAV-2 is provided in GenBank Accession No. NC_{—}001401 and Srivastava et al., J. Virol., 45: 555-564 {1983); the complete genome of AAV-3 is provided in GenBank Accession No. NC_{—}1829; the complete genome of AAV-4 is provided in GenBank Accession No. NC_{—}001829; the AAV-5 genome is provided in GenBank Accession No. AF085716; the complete genome of AAV-6 is provided in GenBank Accession No. NC_{—}00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13 (1): 67-76 (2006); and the AAV-11 genome is provided in Virology, 330 (2): 375-383 (2004).

In another aspect, the invention provides DNA plasmids comprising rAAV genomes of the invention. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10 and AAV-11. Production of pseudotyped rAAV is disclosed in, for example, WO 01/83692 which is incorporated by reference herein in its entirety. In various embodiments, AAV capsid proteins may be modified to enhance delivery of the recombinant vector. Modifications to capsid proteins are generally known in the art. See, for example, US 20050053922 and US 20090202490, the disclosures of which are incorporated by reference herein in their entirety.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senaphthy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. No. 5,786,211; U.S. Pat. No. 5,871,982; and U.S. Pat. No. 6,258,595. The foregoing documents are hereby incorporated by reference in their entirety herein, with particular emphasis on those sections of the documents relating to rAAV production.

The invention thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

In other embodiments, the invention provides rAAV (i.e., infectious encapsidated rAAV particles) comprising a rAAV genome of the invention. In one aspect of the invention, the rAAV genome is a self-complementary genome.

In another aspect, the invention includes, but is not limited to, the exemplified rAAV named “rAAV SMN.” The rAAV SMN genome has in sequence an AAV2 ITR, the chicken β-actin promoter with a cytomegalovirus enhancer, an SV40 intron, the SMN coding DNA set out in SEQ ID NO: 1 (GenBank Accession Number NM_{—}000344.2), a polyadenylation signal sequence from bovine growth hormone and another AAV2 ITR. Conservative nucleotide substitutions of SMN DNA are also contemplated (e.g., a guanine to adenine change at position 625 of GenBank Accession Number NM_{—}000344.2). The genome lacks AAV rep and cap DNA, that is, there is no AAV rep or cap DNA between the ITRs of the genome. SMN polypeptides contemplated include, but are not limited to, the human SMN1 polypeptide set out in NCBI protein database number NP_{—}000335.1. Also contemplated is the SMN1-modifier polypeptide plastin-3 (PLS3) [Oprea et al., Science 320 (5875): 524-527 (2008)]. Sequences encoding other polypeptides may be substituted for the SMN DNA.

The rAAV may be purified by methods standard in the art such as by column chromatography or cesium chloride gradients. Methods for purifying rAAV vectors from helper virus are known in the art and include methods disclosed in, for example, Clark et al., Hum, Gene Ther., 10 (6): 1031-1039 (1999); Schenpp and Clark, Methods Mol. Med., 69 427-443 (2002); U.S. Pat. No. 6,566,118 and WO 98/09657.

In another embodiment, the invention contemplates compositions comprising rAAV of the present invention. These compositions may be used to treat lower motor neuron diseases. In one embodiment, compositions of the invention comprise a rAAV encoding a SMN polypeptide. In other embodiments, compositions of the present invention may include two or more rAAV encoding different polypeptides of interest.

Compositions of the invention comprise rAAV in a pharmaceutically acceptable carrier. The compositions may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Titers of rAAV to be administered in methods of the invention will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg). Dosages may also vary based on the timing of the administration to a human. These dosages of rAAV may range from about 1×10¹¹ vg/kg, about 1×10¹², about 1×10¹³, about 1×10¹⁴, about 1×10¹⁵, about 1×10¹⁶ or more viral genomes per kilogram body weight in an adult. For a neonate, the dosages of rAAV may range from about 1×10¹¹, about 1×10¹², about 3×10¹², about 1×10¹³, about 3×10¹³, about 1×10¹⁴, about 3×10¹⁴, about 1×10¹⁵, about 3×10¹⁵, about 1×10¹⁶, about 3×10¹⁶ or more viral genomes per kilogram body weight.

Methods of transducing nerve or glial target cells with rAAV are contemplated by the invention. The methods comprise the step of administering an intravenous effective dose, or effective multiple doses, of a composition comprising a rAAV of the invention to an animal (including a human being) in need thereof. If the dose is administered prior to development of a disorder/disease, the administration is prophylactic. If the dose is administered after the development of a disorder/disease, the administration is therapeutic. In embodiments of the invention, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. Examples of disease states contemplated for treatment by methods of the invention are listed herein above.

Combination therapies are also contemplated by the invention. Combination as used herein includes both simultaneous treatment or sequential treatments. Combinations of methods of the invention with standard medical treatments (e.g., riluzole in ALS) are specifically contemplated, as are combinations with novel therapies.

Route(s) of administration and serotype(s) of AAV components of rAAV (in particular, the AAV ITRs and capsid protein) of the invention may be chosen and/or matched by those skilled in the art taking into account the infection and/or disease state being treated and the target cells/tissue(s). While delivery to an individual in need thereof after birth is contemplated, intrauteral delivery and delivery to the mother are also contemplated.

Compositions suitable for systemic use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin, and Tween family of products (e.g., Tween 20).

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Transduction with rAAV may also be carried out in vitro. In one embodiment, desired target cells are removed from the subject, transduced with rAAV and reintroduced into the subject. Alternatively, syngeneic or xenogeneic cells can be used where those cells will not generate an inappropriate immune response in the subject.

Suitable methods for the transduction and reintroduction of transduced cells into a subject are known in the art. In one embodiment, cells can be transduced in vitro by combining rAAV with the cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by injection into the spinal cord.

Transduction of cells with rAAV of the invention results in sustained expression of polypeptide. The present invention thus provides methods of administering/delivering rAAV (e.g., encoding SMN protein) of the invention to an animal or a human patient. These methods include transducing nerve and/or glial cells with one or more rAAV of the present invention. Transduction may be carried out with gene cassettes comprising tissue specific control elements. For example, promoters that allow expression specifically within neurons or specifically within astrocytes. Examples include neuron specific enolase and glial fibrillary acidic protein promoters. Inducible promoters under the control of an ingested drug may also be developed.

It will be understood by one of ordinary skill in the art that a polynucleotide delivered using the materials and methods of the invention can be placed under regulatory control using systems known in the art. By way of non-limiting example, it is understood that systems such as the tetracycline (TET on/off) system [see, for example, Urlinger et al., Proc. Natl. Acad. Sci. USA 97 (14):7963-7968 (2000) for recent improvements to the TET system] and Ecdysone receptor regulatable system [Palli et al., Eur J. Biochem 270: 1308-1315 (2003] may be utilized to provide inducible polynucleotide expression. It will also be understood by the skilled artisan that combinations of any of the methods and materials contemplated herein may be used for treating a neurodegenerative disease.

The term “transduction” is used to refer to the administration/delivery of SMN DNA to a recipient cell either in vivo or in vitro, via a replication-deficient rAAV of the invention resulting in expression of a functional SMN polypeptide by the recipient cell.

Thus, the invention provides methods of administering an effective dose (or doses, administered essentially simultaneously or doses given at intervals) of rAAV of the invention to a patient in need thereof.

In still other embodiments, methods of the invention may be used to deliver polynucleotides to a vascular endothelial cell rather than across the BBB.

### BRIEF DESCRIPTION OF THE DRAWINGS

FIG. 1 depicts GFP expression in the gastrocnemius muscle of AAV9-GFP or PBS treated mice.

FIG. 2 depicts widespread neuron and astrocyte AAV9-GFP transduction in CNS and PNS 10-days-post-intravenous injection of postnatal day 1 (P1) mice. (A-B) GFP and ChAT immunohistochemistry of cervical (A) and lumbar (B) spinal cord. (C) High-power magnification shows extensive co-localization of GFP and ChAT positive cells. (arrow indicates GFP-positive astrocyte). (D) Neurons and astrocytes transduced in the hippocampus. (E) Pyramidal cells in the cortex were GFP positive. (F) Clusters of GFP positive astrocytes were observed throughout the brain. Scale bars (A-B) 200 μm, (C) 50 μm, (D-F) 50 μm.

FIG. 3 shows that intravenous injection of AAV9 leads to widespread neonatal spinal cord transduction. Cervical (a-c) and lumbar (e-k) spinal cord sections ten-days following facial-vein injection of 4×10¹¹ particles of scAAV9-CB-GFP into postnatal day-1 mice. GFP-expression (a,e,i) was predominantly restricted to lower motor neurons (a,e,i) and fibers that originated from dorsal root ganglia (a,e). GFP-positive astrocytes (i) were also observed scattered throughout the tissue sections. Lower motor neuron and astrocyte expression were confirmed by co-localization using choline acetyl transferase (ChAT) (b,f,j) and glial fibrillary acidic protein (GFAP) (c,g,k), respectively. A z-stack image (i-k) of the area within the box in h, shows the extent of motor neuron and astrocyte transduction within the lumbar spinal cord. Scale bars, 200 μm (d,h), 20 μm (l).

FIG. 4 shows that intravenous injection of AAV9 leads to widespread and long term neonatal spinal cord transduction in lumbar motor neurons. Z-series confocal microscopy showing GFP-expression in 21-day-old mice that received 4×10¹¹ particles of scAAV9-CB-GFP intravenous injections on postnatal day-1. Z-stack images of GFP (a), ChAT (b), GFAP (c) and merged (d) demonstrating persistent GFP-expression in motor neurons and astrocytes (d) for at least three-weeks following scAAV9-CB-GFP injection. Scale bar, 20 μm (d).

FIG. 5 depicts in situ hybridization of spinal cord sections from neonate and adult injected animals demonstrates that cells expressing GFP are transduced with scAAV9-CB-GFP. Negative control animals injected with PBS (a-b) showed no positive signal. However, antisense probes for GFP demonstrated strong positive signals for both neonate (c) and adult (e) sections analyzed. No positive signals were found for the sense control probe in neonate (d) or adult (f) spinal cord sections. Tissues were counterstained with Nuclear Fast Red for contrast while probe hybridization is in black.

FIG. 6 depicts cervical (A), thoracic (B) and lumbar (C) transverse sections from mouse spinal cord labeled for GFP and ChAT. The box in (C) denotes the location of (D-F). GFP (D), chAT (E) and merged (F) images of transduced motor neurons in the lumbar spinal cord. In addition to motor neuron transductions, GFP positive fibers are seen in close proximity and overlapping motor neurons (D and F). Scale bars=(A-C) 200 μm and (F) 50 μm.

FIG. 7 depicts GFP (A), ChAT (B) and merged (C) images of a transverse section through lumbar spinal cord of a postnatal day 10 (P10) mouse that had previously been injected at one day old with scAAV9 GFP. (D) represents a z-stack merged image of the ventral horn from (C). (E) shows that the scAAV9 vector resulted in more transduced motor neurons when compared to ssAAV9 vector in the lumbar spinal cord. Scale bars=(C) 100 μm and (D) 50 μm.

FIG. 8 depicts AAV9-GFP targeting of astrocytes in the spinal cord of adult-mice. (A-B) GFP immunohistochemistry in cervical (A) and lumbar (B) spinal cord demonstrating astrocyte transduction following tail-vein injection. (hatched-line indicates grey-white matter interface). (C) GFP and GFAP immunohistochemistry from lumbar spinal cord indicating astrocyte transduction. Scale bars (A-B) 100 μm, (C) 20 μm.

FIG. 9 shows that intravenous injection of AAV9 leads to widespread predominant astrocyte transduction in the spinal cord and brain of adult mice. GFP-expression in the cervical (a-c) and lumbar (c-g) spinal cord as well as the brain (m-o) of adult mice 7-weeks after tail vein injection of 4×10¹² particles of scAAV9-CB-GFP. In contrast to postnatal day-1 intravenous injections, adult tail vein injection resulted in almost exclusively astrocyte transduction. GFP (a,e), ChAT (b,f) and GFAP (c,g) demonstrate the abundance of GFP expression throughout the spinal grey matter, with lack of co-localization with lower motor neurons and white matter astrocytes. Co-localization of GFP (i), excitatory amino acid transporter 2 (EAAT2) (j), and GFAP (k) confirm that transduced cells are astrocytes. Tail vein injection also resulted in primarily astrocyte transduction throughout the brain as seen in the cortex (m-n), thalamus (o) and midbrain. Neuronal GFP-expression in the brain was restricted to the hippocampus and dentate gyrus (m-n, FIG. 11e-f).

FIG. 10 depicts diagrams of coronal sections throughout the mouse brain corresponding to the approximate locations shown in (FIG. 9m-o). The box in (a) corresponds to the location shown in (FIG. 9m). The smaller box in (b) corresponds to (FIG. 9n) and the larger box to (FIG. 9o).

FIG. 11 depicts high-magnification of merged GFP and dapi images of brain regions following neonate (a-d) or adult (e-f) intravenous injection of scAAV9-CB-GFP. Astrocytes and neurons were easily detected in the striatum (a), hippocampus (b) and dentate gyrus (c) following postnatal day-1 intravenous injection of 4×10¹¹ particles of scAAV9-CB-GFP. Extensive GFP-expression within cerebellar Purkinje cells (d) was also observed. Pyramidal cells of the hippocampus (e) and granular cells of the dentate gyrus (f) were the only neuronal transduction within the brain following adult tail vein injection. In addition to astrocyte and neuronal transduction, widespread vascular transduction (t) was also seen throughout all adult brain sections examined. Scale bars, 200 μm (e); 100 μm (f), 50 μm (a-d).

FIG. 12 depicts widespread GFP-expression 21-days following intravenous injection of 4×10¹¹ particles of scAAV9-CB-GFP to postnatal day-1 mice. GFP localized in neurons and astrocytes throughout multiple structures of the brain as depicted in: (a) striatum (b) cingulate gyrus (c) fornix and anterior commissure (d) internal capsule (e) corpus callosum (f) hippocampus and dentate gyrus (g) midbrain and (h) cerebellum. All panels show GFP and DAPI merged images. Schematic representations depicting the approximate locations of each image throughout the brain are shown in (FIG. 13). Higher magnification images of select structures are available in (FIG. 11, 14). Scale bars, 200 μm (a); 50 μm (e); 100 μm (b-d,f-h).

FIG. 13 depicts diagrams of coronal sections throughout the mouse brain. corresponding to the approximate locations shown in FIG. 12(a-h) for postnatal day-1 injected neonatal mouse brains. The box in (a) corresponds to the location of (FIG. 12a). The smaller box in (b) corresponds to (FIG. 12b) and the larger box to (FIG. 12c). The larger box in (c) corresponds to (FIG. 12d) while the smaller box in (c) represents (FIG. 12e). Finally, (d-f) correspond to (FIG. 12 f-h) respectively.

FIG. 14 depicts co-localization of GFP positive cells with GAD67. Immunohistochemical detection of GFP (a,d,g,j) and GAD67 (b,e,h,k) expression within select regions of mouse brain 21-days following postnatal day-1 injection of 4×10¹¹ particles of scAAV9-CB-GFP. Merged images (c,f,i,l) show limited co-localization of GFP and GAD67 signals in the cingulate gyrus (a-c), the dentate gyrus (d-f) and the hippocampus (g-i), but numerous GFP/GAD67 Purkinje cells within the cerebellum (l). Scale bars, 100 μm (c), 50 μm (a-b,d-l).

FIG. 15 depicts gel electrophoresis and silver staining of various AAV9-CBGFP vector preparations demonstrates high purity of research grade virus utilized in studies. Shown are 2 vector batches at varying concentrations demonstrating the predominant 3 viral proteins (VP); VP1, 2, 3 as the significant components of the preparation. 1 μl, 5 μl, and 10 μl were loaded of each respective batch of virus.

FIG. 16 depicts direct injection of scAAV9-CB-GFP into the brain and demonstrates predominant neuronal transduction. Injection of virus into the striatum (a) and hippocampus (b) resulted in the familiar neuronal transduction pattern as expected. Co-labeling for GFP and GFAP demonstrate a lack of astrocyte transduction in the injected structures with significant neuronal cell transduction. Scale bars, 50 μm (a), 200 μm (b).

### DETAILED DESCRIPTION

The present invention is illustrated by the following examples relating to a novel rAAV9 and its ability to efficiently deliver genes to the spinal cord via intravenous delivery in both neonatal animals and in adult mice. Example 1 describes experiments showing that rAAV9 can transduce and express protein in mouse skeletal muscle. Example 2 describes experiments in which the expression of the rAAV9 transgene was examined. Example 3 describes the ability of rAAV9 to transduce and express protein in lumbar motor neurons (LMNs). Example 4 describes the evaluation of vectors that do not require second-strand synthesis. Example 5 describes experiments focused on examining whether rAAV9 vectors were enhanced for retrograde transport to target dorsal root ganglion (DRG) and LMNs or could easily pass the blood-brain-barrier (BBB) in neonates. Example 6 describes the evaluation of optimal delivery of rAAV9 expressing SMN for postnatal gene replacement in a mouse model of Type 2 SMA for function and survival. Example 7 describes the examination of the brains of mice following postnatal day-one intravenous injection of scAAV9-CBGFP. Example 8 describes the investigation of whether astrocyte transduction is related to vector purity or delivery route. Example 9 describes administration of scAAV9-GFP in a nonhuman primate.

### Example 1

The ability of AAV9 to target and express protein in skeletal muscle was evaluated in an in vivo model system.

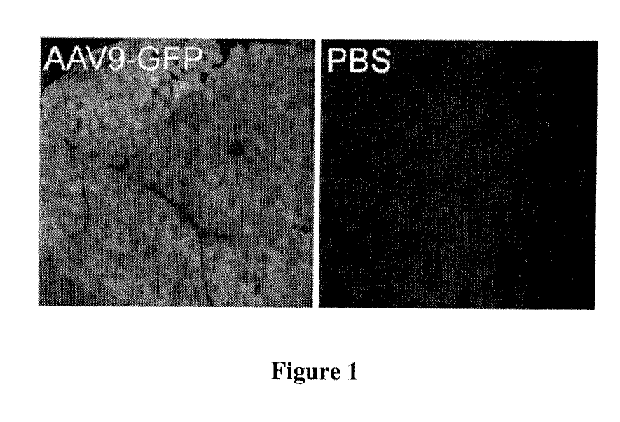

Intravenous administration of 1×10¹¹ particles of scAAV9-GFP was performed in a total volume of 50 μl to postnatal day 1 mice and the extent of muscle transduction was evaluated. The rAAV GFP genome included in sequence an AAV2 ITR, the chicken β-actin promoter, with a cytomegalovirus enhancer, an SV40 intron, the GFP DNA, a polyadenylation signal sequence from bovine growth hormone and another AAV2 ITR. The ability of the AAV9 vectors to transduce skeletal muscle was evaluated using a GFP expressing vector. AAV9-GFP expressed at high levels in the skeletal muscles that were analyzed. Ten (lays following injections, animals were euthanized and gastrocnemius muscles were rapidly isolated, frozen using liquid nitrogen chilled isopentane, and sectioned on a cryostat at 15 μm. Analysis of muscle sections using a Zeiss Axiovert microscope equipped with GFP fluorescence demonstrated that AAV9-GFP expressed at very high levels, with over 90% of the analyzed gastrocnemius muscle transduced (FIG. 1). No GFP expression was detected in PBS control treated animals (FIG. 1). These results showed that AAV9 was effective at targeting and expressing in skeletal muscles.

### Example 2

Transgene expression following intravenous injection in neonatal animals prior to the closure of the BBB and in adult animals was examined.

Mice used were C57B1/6 littermates. The mother (singly housed) of each litter to be injected was removed from the cage. The postnatal day 1 (P1) pups were rested on a bed of ice for anesthetization. For neonate injections, a light microscope was used to visualize the temporal vein (located just anterior to the ear). Vector solution was drawn into a 3/10 cc 30 gauge insulin syringe. The needle was inserted into the vein and the plunger was manually depressed. Injections were in a total volume of 100 μl of a phosphate buffered saline (PBS) and virus solution. A total to of 1×10¹¹ DNase resistant particles of scAAV9 CB GFP (Virapur LLC, San Diego) were injected. One-day-old wild-type mice received temporal vein injections of 1×10¹¹ particles of a self-complementary (sc) AAV9 vector [McCarty et al., Gene therapy, 10: 2112-2118 (2003)] that expressed green fluorescent protein (GFP) under control of the chicken-β-actin hybrid promoter (CB). A correct injection was verified by noting blanching of the vein. After the injection pups were returned to their cage. When the entire litter was injected, the pups were rubbed with bedding to prevent rejection by the mother. The mother was then reintroduced to the cage. Neonate animals were sacrificed ten days post injection, spinal cords and brains were extracted rinsed in PBS, then immersion fixed in a 4% paraformaldehyde solution.

Adult tail vein injections were performed on ˜70 day old C57B1/6 mice. Mice were placed in restraint that positioned the mouse tail in a lighted, heated groove. The tail was swabbed with alcohol then injected intravenously with a 100 μl viral solution containing a mixture of PBS and 5×10¹¹ DNase resistant particles of scAAV9 CB GFP. After the injection, animals were returned to their cages. Two weeks post injection, animals were anesthetized then transcardially perfused first with 0.9% saline then 4% paraformaldehyde. Brains and spinal cords were harvested and immersion fixed in 4% paraformaldehyde for an additional 24-48 hours.

Neonate and adult brains were transferred from paraformaldehyde to a 30% sucrose solution for cryoprotection. The brains were mounted onto a sliding microtome with Tissue-Tek O.C.T. compound (Sakura Finetek USA, Torrance, Calif.) and frozen with dry ice. Forty micron thick sections were divided into 5 series for histological analysis. Tissues for immediate processing were placed in 0.01 M PBS in vials. Those for storage were placed in antifreeze solution and transferred to −20° C. Spinal cords were cut into blocks of tissue 5-6 mm in length, then out into 40 micron thick transverse sections on a vibratome. Serial sections were kept in a 96 well plate that contained 4% paraformaldehyde and were stored at 4° C.

Brains and spinal cords were both stained as floating sections. Brains were stained in a 12-well dish, and spinal cords sections were stained in a 96-well plate to maintain their rostral-caudal sequence. Tissues were washed three times for 5 minutes each in PBS, then blocked in a solution containing 10% donkey serum and 1% Triton X-100 for two hours at room temperature. After blocking, antibodies were diluted in the blocking solution at 1:500. The primary antibodies used were as follows: goat anti-ChAT and mouse anti-NeuN (Chemicon), rabbit anti-GFP (Invitrogen) and guinea pig anti-GFAP (Advanced Immunochemical). Tissues were incubated in primary antibody at 4° C. for 48-72 hours then washed three times with PBS. After washing, tissues were incubated for 2 hours at room temperature in the appropriate secondary antibodies (1:125 Jackson Immunoresearch) with DAPI. Tissues were then washed three times with PBS, mounted onto slides then coverslipped. All images were captured on a Zeiss laser-scanning eon focal microscope.

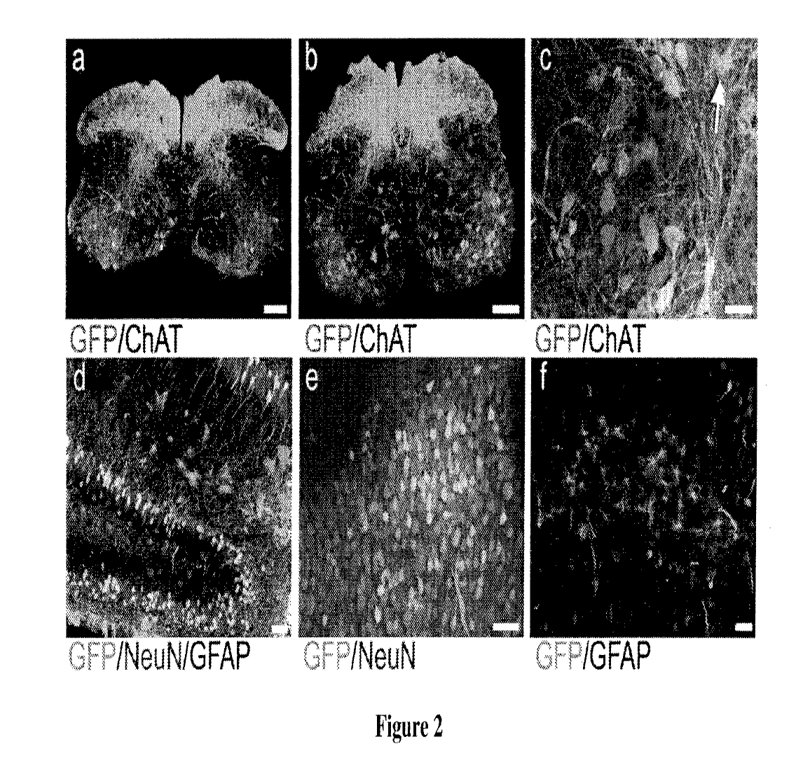

Spinal cords had remarkable GFP expression throughout all levels with robust GFP expression in fibers that ascended in the dorsal columns and fibers that innervated the spinal gray matter, indicating dorsal root ganglia (DRG) transduction. GFP positive cells were also found in the ventral region of the spinal cord where lower motor neurons reside (FIG. 2A-B). Labeling of choline acetyl transferase (ChAT) positive cells with GFP demonstrated a large number of ChAT positive cells expressing GFP throughout all cervical and lumbar sections examined, indicating widespread LMN transduction (FIG. 2C). Approximately 56% of ChAT positive cells strongly expressed GFP in sections analyzed of the lumbar spinal cord (598 GFP+/1058 ChAT+, n=4) (Table 1, below). This is the highest proportion of LMNs transduced by a single injection of AAV reported. Stereology for total number of neurons in a given area and total number of GFP+ cells was performed on a Nikon E800 fluorescent microscope with computer-assisted microscopy and image analysis using StereoInvestigator software (MicroBrightField, Inc., Williston, Vt.) with the optical dissector principle to avoid oversampling errors and the Cavalieri estimation for volumetric measurements. Coronal 40 μm sections, 240 μm apart covering the regions of interest in its rostro-caudal extension was evaluated. The entire dentate gyrus, caudal retrosplenial/cingulate cortex; containing the most caudal extent of the dentate gyrus; extending medially to the subiculum and laterally to the occipital cortex, and the purkinje cell layer was sampled using ˜15-25 optical dissectors in each case. Fluorescent microscopy using a 60× objective for NeuN and GFP were utilized and cells within the optical dissector were counted on a computer screen. Neuronal density and positive GFP density were calculated by multiplying the total volume to estimate the percent of neuronal transduction in each given area as previously described [Kempermann et al., Proceedings of the National Academy of Sciences of the United States of America 94: 10409-10414 (1997)].

For motor neuron quantification, serial 40 μm thick lumbar spinal cord sections, each separated by 480 μm, were labeled as described for GFP and ChAT expression. Stained sections were serially mounted on slides from rostral to caudal, then coverslipped. Sections were evaluated using confocal microscopy (Zeiss) with a 40× objective and simultaneous FITC and Cy3 filters. FITC was visualized through a 505-530 nm band pass filter to avoid contaminating the Cy3 channel. The total number of ChAT positive cells found in the ventral horns with defined soma was tallied by careful examination through the entire z-extent of the section. GFP labeled cells were quantified in the same manner, while checking for co-localization with ChAT. The total number of cells counted per animal ranged from approximately 150-366 cells per animal. For astrocyte quantification, as with motor neurons, serial sections were stained for GFP, GFAP and EAAT2, then mounted. Using confocal microscopy with a 63× objective and simultaneous FITC and Cy5 filters, random fields in the ventral horns of lumbar spinal cord sections from tail vein injected animals were selected. The total numbers of GFP and GFAP positive cells were counted from a minimum of at least 24-fields per animal while focusing through the entire z extent of the section.

In addition to widespread DRG and motor neuron transduction, GFP-positive glial cells were observed throughout the spinal gray matter (FIG. 2C; arrow). The brains were next examined following P1 intravenous injection of AAV9-CB-GFP and revealed extensive GFP expression in all regions analyzed, including the hippocampus (FIG. 2D), cortex (FIG. 2E), striatum, thalamus, hypothalamus and choroid plexus, with predominant neuronal transduction. However, transduced astrocytes were also found in all regions of the brain examined (FIG. 2F).

The remarkable pattern of GFP expression observed following P1 administration suggests two independent modes of viral entry into the central nervous system (CNS). Due to the ubiquitous GFP expression throughout the brain, the virus likely crossed the developing BBB. However the GFP expression pattern in the neonate spinal cord is defined with respect to the specific DRG and LMN transduction. The DRG and the LMN have projections into the periphery which suggests retrograde transport may be the mechanism of transduction. In support of retrograde transport as the method of spinal cord neuronal transduction, there were no GFP positive interneurons observed in any section examined. Alternatively, the virus may have a LMN tropism after crossing the BBB, but this appears unlikely as ChAT positive cells still migrating from the central canal to the ventral horn were largely untransduced (FIG. 2A-B).

TABLE 1

| | |
|---|---|
| GFP (mean +/− s.e.m.) | % (mean +/− s.e.m.) |

Neonate

| | | | | |
|---|---|---|---|---|
| | | | NeuN (mean +/− s.e.m.) | |
| Brain | Retrosplental/Cingulate | 142,658.30 +/− 11124.71 | 762.104.30 +/− 38397.81 | 18.84 +/− 1.93 |
| | Denate Gyrus | 42,304.33 +/− 15613.33 | 278,043.70 +/− 11383.56 | 14.82 +/− 4.89 |
| | Purkinje cells | 52,720.33 +/− 1951.33 | 73,814.66 +/− 5220.80 | 71.88 +/− 3.65 |
| | | | ChAT (mean +/− s.e.m) | |
| Lumbar | 10 days post injection | 149.5 +/− 31.65 | 264.5 +/− 53.72 | 56.18 +/− 1.95 |
| spinal cord | 21 days post injection | 83.33 +/− 16.33 | 140.0 +/− 31.76 | 60.79 +/− 2.96 |

Adult

| | | | | |
|---|---|---|---|---|
| | | | GFAP (mean +/− s.e.m.) | |
| Lumber | % GFP colabeled w/ GFAP | 48.00 +/− 10.12 | 43.00 +/− 7.00 | 91.44 +/− 4.82 |
| spinal cord | % GFAP+ transduced | 41.33 +/− 5.55 | 64.33 +/− 8.67 | 64.23 +/− 0.96 |
| (grey matter) | | | | |

Additional experiments were done on one-day-old wild-type mice where they were administered temporal vein injections of 4×10¹¹ particles of a self-complementary (sc) AAV9 vector [McCarty et al., Gene therapy 10: 2112-2118 (2003)] that expressed green fluorescent protein (GFP) under control of the chicken-β-actin hybrid promoter (CB).

Histological processing was performed as above. Brains and spinal cords were both stained as floating sections. Brains were stained in a 12-well dish, and spinal cords sections were stained in a 96-well plate to maintain their rostral-caudal sequence. Tissues were washed three-times for 5-minutes each in PBS, then blocked in a solution containing 10% donkey serum and 1% Triton X-100 for two hours at room temperature. After blocking, antibodies were diluted in the blocking solution at 1:500. The primary antibodies used were as follows: goat anti-ChAT and mouse anti-NeuN (Millipore, Billerica, Mass.), rabbit anti-GFP (Invitrogen, Carlsbad, Calif.), guinea pig anti-GFAP (Advanced Immunochemical, Long Beach, Calif.) and goat anti-GAD67 (Millipore, Billerica, Mass.). Tissues were incubated in primary antibody at 4° C. for 48-72 hours then washed three times with PBS. After washing, tissues were incubated for 2 hours at room temperature in the appropriate secondary antibodies (1:125 Jackson Immunoresearch, Westgrove, Pa.) with DAPI. Tissues were then washed three times with PBS, mounted onto slides then coverslipped. All images were captured on a Zeiss-laser-scanning confocal microscope.

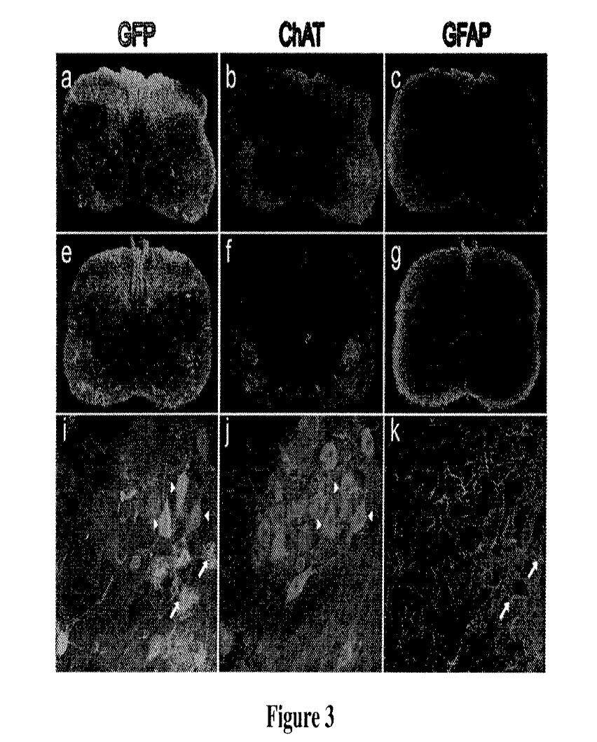

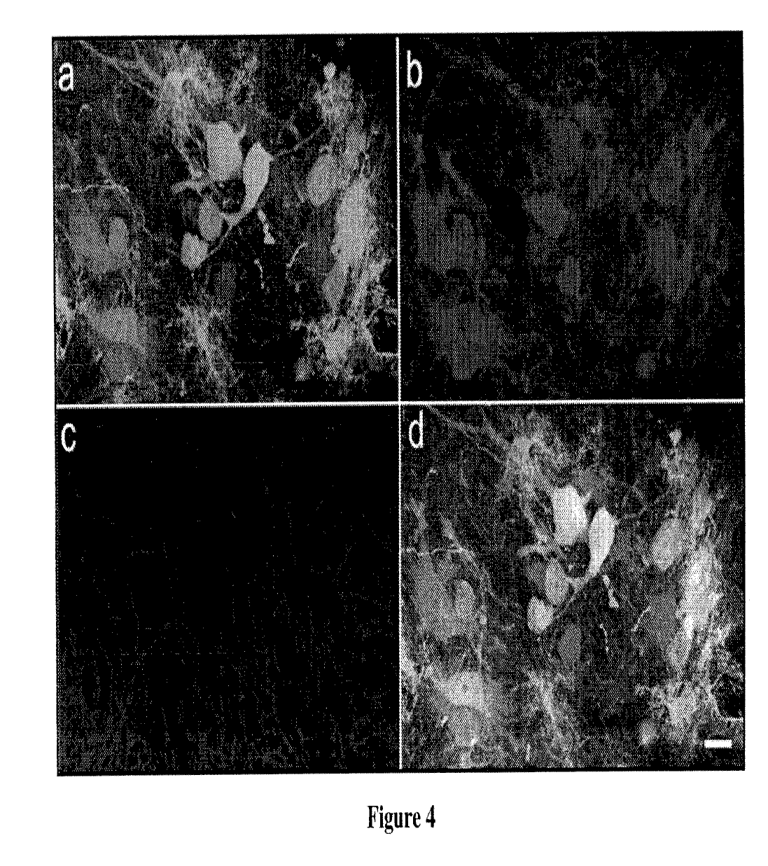

Animals were sacrificed 10- or 21-days post-injection, and brains and spinal cords were evaluated for transgene expression. Robust GFP-expression was found in heart and skeletal muscles as expected. Strikingly, spinal cords had remarkable GFP-expression throughout all levels, with robust GFP-expression in fibers that ascended in the dorsal columns and fibers that innervated the spinal grey matter, indicating dorsal root ganglia (DRG) transduction. GFP-positive cells were also found in the ventral region of the spinal cord where lower motor neurons reside (FIG. 3a and e). Co-labeling for choline acetyl transferase (ChAT) and GFP-expression within the spinal cord demonstrated a large number of ChAT positive cells expressing GFP throughout all cervical and lumbar sections examined, indicating widespread LMN transduction (FIG. 4). Approximately 56% of ChAT positive cells strongly expressed GFP in sections analyzed of the lumbar spinal cord of 10 day-old animals and ˜61% of 21 day-old animals, demonstrating early and persistent transgene expression in lower motor neurons (Table 1). Similar numbers of LMN expression were seen in cervical and thoracic regions of the spinal cord. This is the highest proportion of LMNs transduced by a single injection of AAV reported. In addition to widespread DRG and motor neuron transduction, we observed GFP-positive glial cells throughout the spinal grey matter, indicating that AAV9 could express in astrocytes with the CB promoter. The remarkable pattern of GFP-expression observed following postnatal day-one administration suggests two independent modes of viral entry into the CNS. Due to the ubiquitous GFP-expression throughout the brain, the virus likely crossed the developing BBB. However the GFP-expression pattern in the neonate spinal cord is defined with respect to the specific DRG and LMN transduction. The DRG and the LMN have projections into the periphery which suggests retrograde transport may be the mechanism of transduction. In support of retrograde transport as the method of spinal cord neuronal transduction, there were no GFP-positive interneurons observed in any section examined. Alternatively, the virus may have a LMN tropism after crossing the BBB, but this appears unlikely as ChAT positive cells still migrating from the central canal to the ventral horn were largely untransduced.

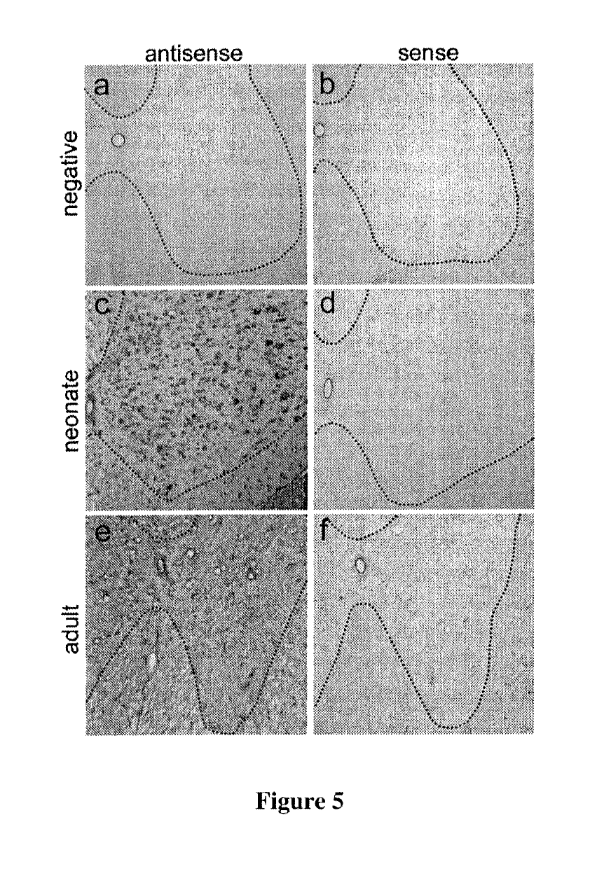

In situ hybridization confirmed that viral transcription, and not protein uptake, was responsible for the previously unseen transduction pattern (FIG. 5).

### Example 3

The ability of AAV9 to transduce and express protein in LMN was evaluated.

LMN transduction in the lumbar ventral horn was evaluated following intravenous administration of 1×10¹¹ particles of ss or scAAV9 GFP to postnatal day 1 mice in an effort to effectively deliver a transgene to spinal cord motor neurons. Both single-stranded and self-complementary AAV9-GFP vectors were produced via transient transfection production methods and were purified two times on CsCl gradients. The AAV9 GFP genomes are identical with the exception that scAAV genomes have a mutation in one ITR to direct packaging of specifically self-complementary virus. The single stranded AAV constructs do not contain the ITR mutation and therefore package predominantly single stranded virus. Viral preps were titered simultaneously using TAQMAN Quantitative PCR. P1 mice (n=5/group) were placed on an ice-cold plates to anesthetize and virus was delivered using 0.3 cc insulin syringes with 31 gauge needles that were inserted into the superficial facial vein. Virus was delivered in a volume of 50 μl. Animals recovered quickly after gene delivery with no adverse events noted. Animals were injected with a xylazine/ketamine mixture and were decapitated 10-days following injection and spinal cords were harvested then post-fixed in 4% paraformaldehyde, sectioned using a Vibratome and immunohistochemistry was performed using co-labeling for ChAT and GFP. Analysis of GFP expression was performed using a Zeiss Confocal Microscope.

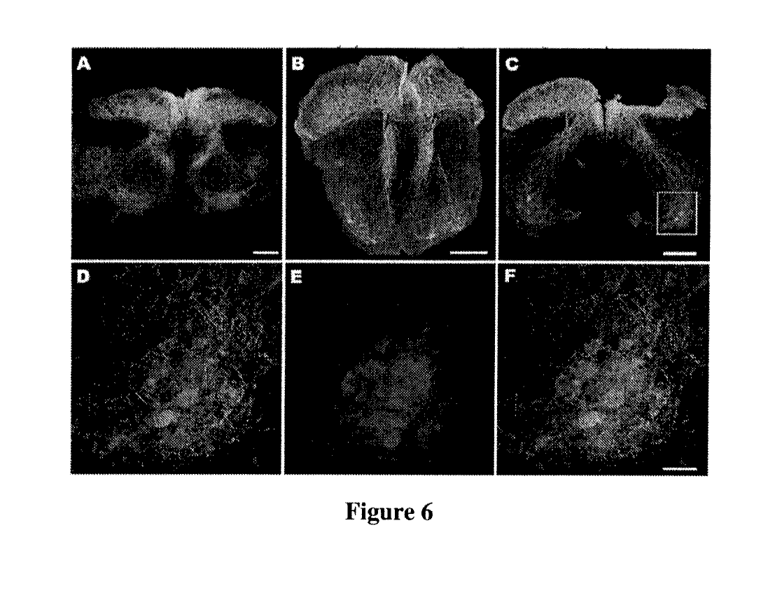

Intravenous injection of single stranded AAV9-GFP resulted in widespread DRG transduction as evidenced by GFP positive fibers innervating the spinal grey matter and ascending in the dorsal columns (FIG. 6A-C). Numerous sections showed strong GFP staining in motor neurons as assessed by co-labeling GFP with Choline acetyltransferase (ChAT) (FIG. 3E-F). Counting the total number of motor neurons in treated animals demonstrated approximately 8% of total motor neurons residing in the lumbar region of the spinal cord were transduced. This finding was remarkable given that motor neuron transduction has typically been very low (less than 1% of total motor neurons), particularly by remote delivery approaches such as retrograde transport.

### Example 4

Self-complementary scAAV9 vectors that do not require second-strand synthesis (a rate limiting step of AAV vectors) which would allow for greater efficiencies of expression in motor neurons, were evaluated.

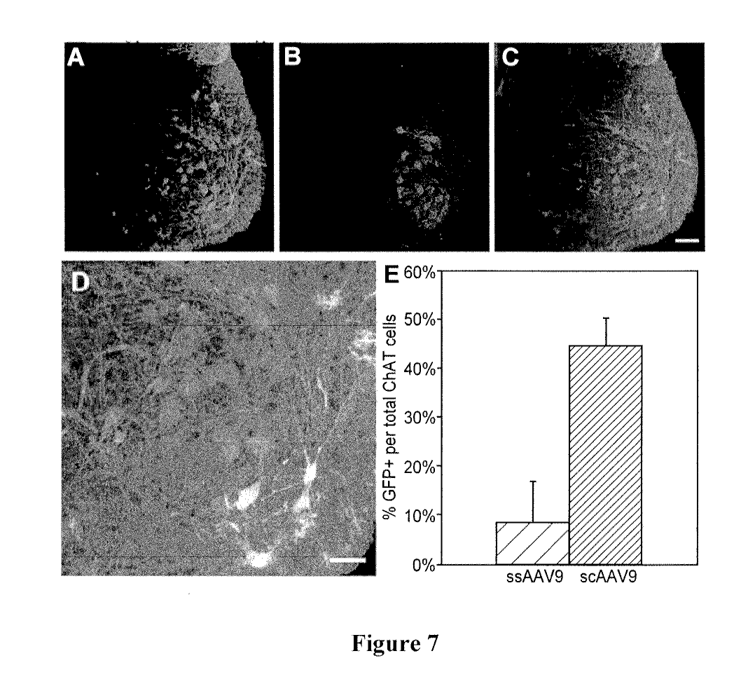

Viral particles were prepared as in Example 3. Intravenous injections into the facial vein of P1 pups were performed as described above and the animals as described above 10 days post-injection. As with ssAAV9 injections significant transduction of DRG was observed throughout the spinal cord. Remarkably, significant motor neuron transduction in treated animals was found in the two areas of the spinal cord that were evaluated including the cervical and lumbar spinal cord. Quantification of GFP+/ChAT+ double labeled cells expressed as a percentage of total ChAT+ cells within the lumbar spinal cord showed that ˜45% of LMN were transduced by dsAAV9 compared with ˜8% of ssAAV9 (FIG. 7E). Indeed, some regions of the spinal cord showed >90% motor neuron transduction (FIG. 7D) and other regions may have greater amounts of GFP positive motor neurons, given that dim GFP positive cells were not counted due to a conservative GFP positive scoring used in the counting. This amount of LMN transduction following a single injection of AAV has not previously been reported.

### Example 5

Further investigation focused on whether AAV9 vectors were enhanced for retrograde transport to target DRG and LMNs or could easily pass the BBB in neonates.

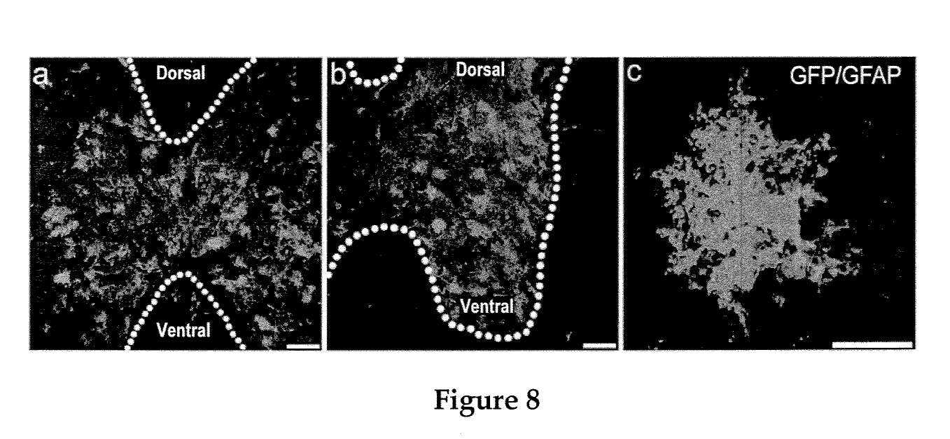

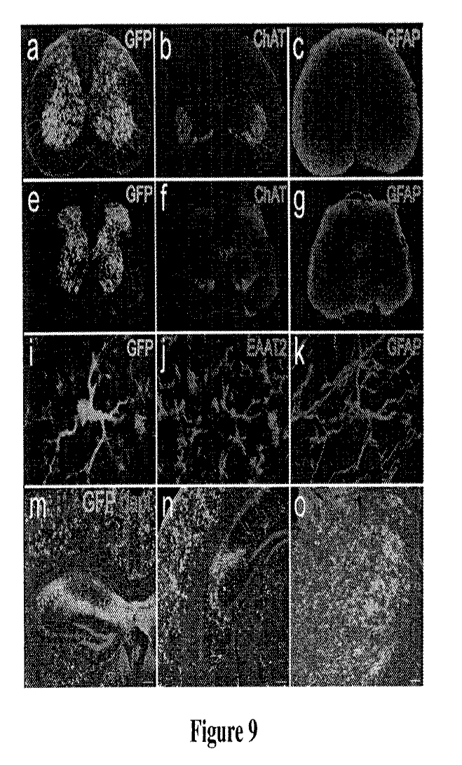

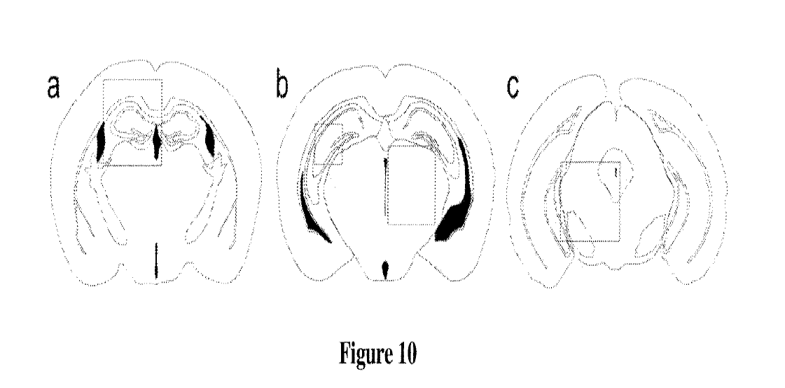

The pattern of transduction was examined to determine if it was consistent between neonates and adult animals. Adult mice were injected via tail vein delivery using 4×10¹¹ to 5×10¹¹ particles of scAAV9-CB-GFP. A strikingly different transduction pattern was seen in adult treated animals compared to the treated neonates. Most noticeably, there was an absence of GFP positive DRG fibers and a marked decrease in LMN transduction in all cervical and lumbar spinal cord sections examined. GFP-positive astrocytes were easily observed throughout the entire dorsal-ventral extent of the grey matter in all regions of the spinal cord (FIG. 8a-b and FIG. 9a-c and e-g) with the greatest GFP-expression levels found in the higher dosed animals. Co-labeling of GFP-positive cells with astroglial markers excitatory amino acid transporter 2 (EAAT2) and glial fibrillary acidic protein (GFAP) (FIG. 8C) demonstrated that approximately 90% of the GFP-positive cells were astrocytes. Counts of total astrocytes in the lumbar region of the spinal cord by z-series collected confocal microscopy showed over 64% of total astrocytes were positive for GFP (FIG. 9i-k and Table 1). FIG. 10 depicts diagrams of coronal sections throughout the mouse brain corresponding to the approximate locations shown in (FIG. 9m-o). The box in (a) corresponds to the location shown in (FIG. 9m). The smaller box in (b) corresponds to (FIG. 9n) and the larger box to (FIG. 9o).

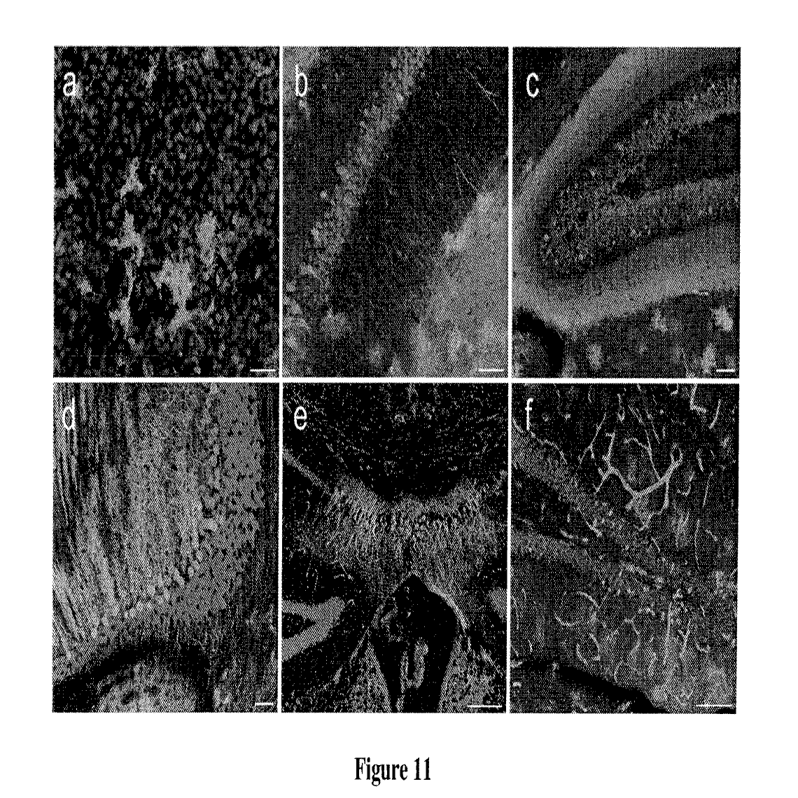

Viral transcription was again confirmed in adult tissues with in situ hybridization (FIG. 5). Furthermore, whereas neonate intravenous injection resulted in indiscriminate astrocyte and neuronal transduction throughout the brain, adult tail-vein injections produced isolated and localized neuronal expression only in the hippocampus and dentate gyrus (FIG. 9m-n and FIG. 11e-f) in both low and high dose animals. Low-dose animals had isolated patches of transduced astrocytes scattered throughout the entire brain. Of significance, high-dose animals had extensive astrocyte and vascular transduction throughout the entire brain (FIG. 9m-o and FIG. 11e-f) that persisted for at least seven-weeks post-injection (n=5), suggesting a dose-response of transduction, without regional specificity.

To date, efficient glial transduction has not been reported for any AAV serotype indicating that AAV9 has a unique transduction property in the CNS following intravenous delivery. An occasional neuron transduced in the spinal cord, although these events were scarce in adult animals. Furthermore, whereas neonate intravenous injection resulted in indiscriminate transduction throughout the brain, adult tail vein injections produced isolated and localized neuronal expression in the hippocampus with isolated patches of glial transduction scattered throughout the entire brain. The scarcity of LMN and DRG transduction seen in the adult paradigm suggests there is a developmental period in which access by circulating virus to these cell populations becomes restricted. Assuming a dependence on retrograde transport for DRG and LMN transduction following intravenous injection, Schwann cell or synapse maturation may be an important determinant of successful rAAV9 LMN and DRG transduction.

The results demonstrate the striking capacity of AAV9 to efficiently target neurons, and in particular motor neurons in the neonate and astrocytes in the adult following intravenous delivery. A simple intravenous injection of AAV9 as described here is clinically relevant for both SMA and ALS. In the context of SMA, data suggests that increased expression of survival motor neuron (SMN) gene in LMNs may hold therapeutic benefit [Azzouz et al., The Journal of Clinical Investigation, 114: 1726-1731 (2004) and Baughan et al., Mol. Ther. 14: 54-62 (2006)]. The importance of the results presented here is that with a single injection SMN expression levels are effectively restored in LMN. Additionally, given the robust neuronal populations transduced throughout the CNS in neonatal animals, this approach also allows for overexpressing or inhibiting genes using siRNA [see, for example, Siegel et al., PLoS Biology, 2: e419 (2004)]. The results also demonstrated efficient targeting of astrocytes in adult-treated animals and this finding is relevant for treating ALS where the non-cell autonomous nature of disease progression has recently been discovered and astrocytes have been specifically linked to disease progression [Yamanaka et al., Nature Neuroscience, 11: 251-253 (2008)]. Targeting these cells with trophic factors or to circumvent aberrant glial activity is useful in treating ALS [Dodge et al., Mol. Ther., 16 (6):1056-64 (2008)].

### Example 6

Optimal delivery of AAV9 expressing SMN is described for postnatal gene replacement in a mouse model of Type 2 SMA.

Studies of the SMA patient population and the various SMA animal models have established a positive correlation between amounts of full-length SMN protein produced and lessened disease severity. Histone deacetylase (HDAC) inhibitors and small molecules are currently being investigated for their ability to increase transcript production or alter exon 7 inclusion from the remaining SMN2 gene [Avila et al., J. Clin. Invest., 117 (3):659-71 (2007) and Chang et al., Proc. Natl. Acad. Sci. USA, 98 (17):9808-9813 (2001)]. Data presented herein demonstrates that a large percentage of LMNs can be targeted with a scAAV9 vector, and SMN gene replacement to treat SMA animals is therefore contemplated.

Mendelian inheritance predicts 25% of the pups in the litters of SMA breeders to be affected. Affected SMA mice are produced by interbreeding SMN2^{+/+}, SMNΔ7^{+/+}, Smn^{+/-} mice. Breeders are maintained as homozygotes for both transgenes and heterzygotes for the knockout allele. Mice were genotyped by PCR following extraction of total genomic DNA from a tail snip (see below). One primer set was used to confirm the presence of the knockout allele while the second primer set detected an intact mouse Smn allele. Animals were treated with either scAAV9 SMN or scAAV9 GFP as controls.

SMA parent mice (Smn^{+/-}, SMN2^{+/+}, SMNΔ7^{+/+} were time mated [Monani et al., Human Molecular Genetics 9: 333-339 (2000)]. Cages were monitored 18-21 days after visualization of a vaginal plug for the presence of litters. Once litters were delivered, the mother was separated out, pups were given tattoos for identification and tail samples were collected. Tail samples were incubated in lysis solution (25 mM NaOH, 0.2 mM EDTA) at 90° C. for one hour. After incubation, tubes were placed on ice for ten minutes and then received an equal volume of neutralization solution (40 mM Tris pH5). After the neutralization buffer, the extracted genomic DNA was added to two different PCR reactions for the mouse Smn allele (Forward 1: 5′-TCCAGCTCCGGGATATTGGGATTG (SEQ ID NO: 2), Reverse 1: 5′-AGGTCCCACCACCTAAGAAAGCC (SEQ ID NO: 3), Forward 2: 5′-GTGTCTGGGCTGTAGGCATTGC (SEQ ID NO: 4), Reverse 2: 5′-GCTGTGCCTTTTGGCTTATCTG (SEQ ID NO: 5)) and one reaction for the mouse Smn knockout allele (Forward: 5′-GCCTGCGATGTCGGTTTCTGTGAGG (SEQ ID NO: 6), Reverse: 5′-CCAGCGCGGATCGGTCAGACG (SEQ ID NO: 7)). After analysis of the genotyping PCR, litters were culled to three animals. Affected animals (Smn^{-/-}, SMN2^{+/+}, SMNΔ7^{+/+}) were injected as previously described with 5×10¹¹ particles of self complementary AAV9 SMN or GFP [Foust et al., Nat Biotechnol 27: 59-65 (2009)].

AAV9 was produced by transient transfection procedures using a double stranded AAV2-ITR based CB-GFP vector, with a plasmid encoding Rep2Cap9 sequence as previously described [Gao et al., Journal of Virology 78: 6381-6388 (2004)] along with an adenoviral helper plasmid; pHelper (Stratagene, La Jolla, Calif.) in 293 cells. The serotype 9 sequence was verified by sequencing and was identical to that previously described [Gao et al., Journal of Virology 78: 6381-6388 (2004)]. Virus was purified by two cesium chloride density gradient purification steps, dialyzed against phosphate-buffered-saline (PBS) and formulated with 0.001% Pluronic-F68 to prevent virus aggregation and stored at 4° C. All vector preparations were titered by quantitative-PCR using Taq-Man technology. Purity of vectors was assessed by 4-12% SDS-Acrylamide gel electrophoresis and silver staining (Invitrogen, Carlsbad, Calif.).

To determine transduction levels in SMA mice (SMN2^{+/+}; SMNΔ7^{+/+}; Smn^{-/-}), 5×10¹¹ genomes of scAAV9-GFP or -SMN (n=4 per group) under control of the chicken-β-actin hybrid promoter were injected into the facial vein at P1. Forty-two ±2% of lumbar spinal motoneurons were found to express GFP 10 days post injection. The levels of SMN in the brain, spinal cord and muscle in scAAV9-SMN-treated animals are shown in. SMN levels were increased in brain, spinal cord and muscle in treated animals, but were still below controls (SMN2^{+/+}; SMNΔ7^{+/+}; Smn^{+/-}) in neural tissue. Spinal cord immunohistochemistry demonstrated expression of SMN within choline acetyl transferase (ChAT) positive cells after scAAV9-SMN injection.

Pups were weighed daily and tested for righting reflex every other day from P5-P13. Righting reflex is analyzed by placing animals on a flat surface on their sides and timing 30 seconds to evaluate if the animals return to a upright position [Butchbach et al., Neurobiology of Disease 27: 207-219 (2007)]. Every five days between P15 and P30, animals were tested in an open field analysis (San Diego Instruments, San Diego, Calif.). Animals were given several minutes within the testing chamber prior to the beginning of testing then activity was monitored for live minutes. Beam breaks were recorded in the X, Y and Z planes, averaged across groups at each time point and then graphed.

Whether scAAV9-SMN treatment of SMA animals improved motor function was then evaluated. SMA animals treated with scAAV9-SMN or -GFP were evaluated for the ability of the animals to right themselves compared to control and untreated animals (n=10 per group). Control animals were found to right themselves quickly, whereas the SMN- and GFP-treated SMA animals showed difficulty at P5. By P13, however, 90% of SMN treated animals could right themselves compared to 20% of GFP-treated controls and 0% of untreated SMA animals, demonstrating that SMN-treated animals improved. Evaluating animals at P18 showed SMN-treated animals were larger than GFP-treated but smaller than controls. Locomotive ability of the SMN-treated animals were nearly identical to controls as assayed by x, y and z plane beam breaks (open field testing) and wheel running. Age-matched untreated SMA animals were not available as controls for open field or running wheel analysis due to their short lifespan.

Survival in SMN-treated SMA animals (n=11) compared to GFP-treated SMA animals (n=11) was then evaluated using Kaplan Meier survival analysis. No GFP-treated control animals survived past P22, with a median lifespan of 15.5 days. The body weight in treated SMN- or GFP-treated animals compared to wild-type littermates was analyzed. The GFP-treated animal's weight peaked at P10 and then precipitously declined until death. In contrast, SMN-treated animals showed a steady weight in to approximately P40, where the weight stabilized at 17 grams, half of the weight of controls. No deaths occurred in the SMN-treated group until P97. Furthermore, this death appeared to be unrelated to SMA. The mouse died after trimming of long extensor teeth. Four animals (P90-99) were euthanized for electrophysiology of neuromuscular junctions (NMJ). The remaining six animals remain alive, surpassing 250 days of age.

For electrophysiology analysis, a recording chamber was continuously perfused with Ringer's solution containing the following (in mmol/l): 118 NaCl, 3.5 KCl, 2 CaCl₂, 0.7 MgSO₄, 26.2 NaHCO₃, 1.7 NaH₂PO₄, and 5.5 glucose, pH 7.3-7.4 (20-22° C., equilibrated with 95% O₂ and 5% CO₂). Endplate recordings were performed as follows. After dissection, the tibialis anterior muscle was partially bisected and folded apart to flatten the muscle. After pinning, muscle strips were stained with 10 μM 4-Di-2ASP [4-(4-diethylaminostyryl)-Nmethylpyridinium iodide] (Molecular Probes) and imaged with an upright epifluorescence microscope. At this concentration, 4-Di-2ASP staining enabled visualization of surface nerve terminals as well as individual surface muscle fibers. All of the endplates were imaged and impaled within 100 μm. Two-electrode voltage clamp were used to measure endplate current (EPC) and miniature EPC (MEPC) amplitude. Muscle fibers were crushed away from the endplate band and voltage clamped to −45 mV to avoid movement after nerve stimulation.

To determine whether the reduction in endplate currents (EPCs) was corrected with scAAV9-SMN. EPCs were recorded from the tibialis anterior (TA) muscle [Wang et al., J Neurosci 24, 10687-10692 (2004)]. P9-10 animals were evaluated to ensure the presence of the reported abnormalities within our mice. Control mice had an EPC amplitude of 19.1±0.8 nA versus 6.4±0.8 nA in untreated SMA animals (p=0.001) confirming published results [Kong et al., J Neurosci 29, 842-851 (2009)]. Interestingly, P10 scAAV9-SMN-treated SMA animals had a significant improvement (8.8±0.8 vs. 6.4±0.8 nA, p<0.05) over age-matched untreated SMA animals. Gene therapy treatment, however, had not restored normal EPC at P10 (19.1±0.8 vs. 8.8±0.8 nA, p=0.001). At P90-99, there was no difference in EPC amplitude between controls and SMA mice that had been treated with scAAV-SMN. Thus, treatment with scAAV9-SMN fully corrected the reduction in synaptic current. Importantly, P90-99 age-matched untreated SMA animals were not available as controls due to their short lifespan.

The number of synaptic vesicles released following nerve stimulation (quantal content) and the amplitude of the muscle response to the transmitter released from a single vesicle (quantal amplitude) determine the amplitude of EPCs. Untreated SMA mice have a reduction in EPC due primarily to reduced quantal content [Kong et al., J Neurosci 29, 842-851 (2009)]. In our P9-10 cohort, untreated SMA animals had a reduced quantal content when compared with wild-type controls (5.7±10.6 vs. 12.8±0.6, p<0.05), but scAAV9-SMN treated animals were again improved over the untreated animals (9.5±0.6 vs. 5.7±0.6, p<0.05), but not to the level of wild-type animals (9.5±0.6 vs. 12.8±0.6, p<0.05). At P90-99, when quantal content was measured in treated SMA mice, a mild reduction was present (control=61.3±3.5, SMA-treated=50.3±2.6, p<0.05), but was compensated for by a statistically significant increase in quantal amplitude (control=1.39±0.06, SMA treated=1.74±0.08, p<0.05). Quantal amplitudes in young animals had no significant differences (control=1.6±0.1, untreated SMA=1.3±0.1, treated SMA=1.1±0.1 nA, p=0.28).

The reduction in vesicle release in untreated SMA mice was due to a decrease in probability of vesicle release, demonstrated by increased facilitation of EPCs during repetitive stimulation [Kong et al., J Neurosci 29: 842-851 (2009)]. Both control and treated SMA EPCs were reduced by close to 20% by the 10th pulse of a 50 Hz train of stimuli (22±3% reduction in control vs 19±1% reduction in treated SMA, p=0.36). This demonstrates that the reduction in probability of release was corrected by replacement of SMN. During electrophysiologic recording, no evidence of denervation was noted. Furthermore, all adult NMJs analyzed showed normal morphology and full maturity. P9-10 transverse abdominis immunohistochemistry showed the typical neurofilament accumulation in untreated SMA NMJs [Kong et al., J Neurosci 29: 842-851 (2009)], whereas treated SMA NMJs showed a marked reduction in neurofilament accumulation.

A recent study using an HDAC inhibitor to extend survival of SMA mice reported necrosis of the extremities and internal tissues [Narver et al., Ann Neurol 64: 465-470 (2008)]. In the studies described herein, mice developed necrotic pinna between P45-70. Pathological examination of the pinna noted vascular necrosis, but necrosis was not found elsewhere.

To explore the therapeutic window in SMA mice, systemic scAAV9-GFP injections were performed at varying postnatal time points to evaluate the pattern of transduction of motor neurons and astrocytes. scAAV9-GFP systemic injections in mice on P2, P5 or P10 showed distinct differences in the spinal cord. There was a shift from neuronal transduction in P2-treated animals toward predominantly glial transduction in older, P10 animals, consistent with previous studies and knowledge of the developing blood-brain barrier in mice [Foust et al., Nat. Biotechnol. 27: 59-65 (2009); Saunders et al., Nat. Biotechnol. 27: 804-805, author reply 805 (2009)].

To determine the therapeutic effect of SMN delivery at these various time points, small cohorts of SMA-affected mice were injected with scAAV9-SMN on P2, P5 and P10 and evaluated for changes in survival and body weight. P2-injected animals were rescued and indistinguishable from animals injected with scAAV9-SMN on P1. However, P5-injected animals showed a more modest increase in survival of approximately 15 days, whereas P10-injected animals were indistinguishable from GFP-injected SMA pups. These findings support previous studies demonstrating the importance of increasing SMN levels in neurons of SMA mice [Gavrilina et al., Hum. Mol. Genet. 17: 1063-1075 (2008)]. Furthermore, these results suggest a period during development in which intravenous injection of scAAV9 can target neurons in sufficient numbers for benefit in SMA.

The above results demonstrate robust, postnatal rescue of SMA mice with correction of motor function, neuromuscular electrophysiology, and increased survival following a one-time gene delivery of SMN. Intravenous scAAV9 treats neurons, muscle and vascular endothelium. Vascular delivery of scAAV9 SMN in the mouse was safe, and well tolerated.

### Example 7

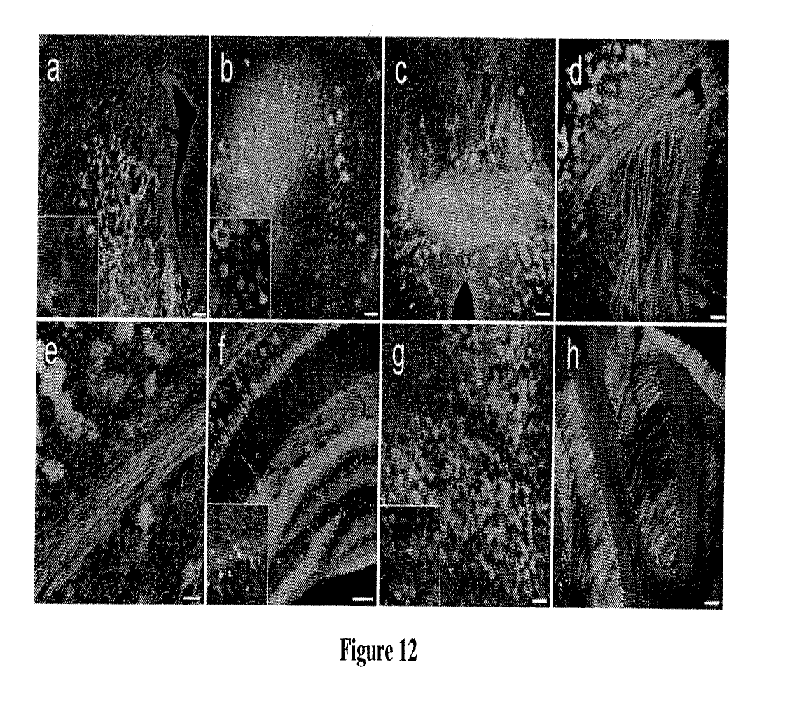

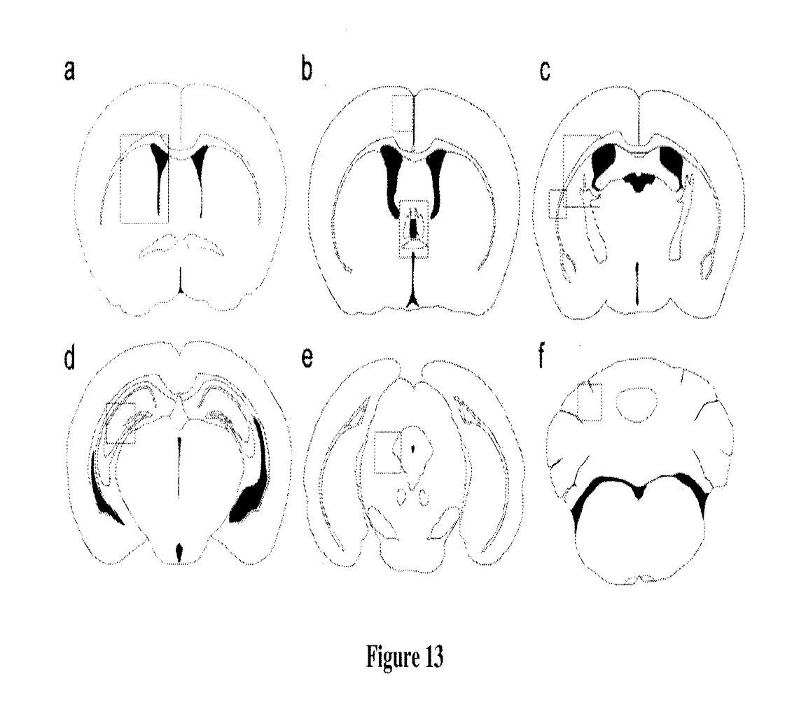

The brains of mice were examined following postnatal day-one intravenous injection of scAAV9-CBGFP and extensive GFP-expression was found in all regions analyzed, including the striatum, cortex, anterior commisure, internal capsule, corpus callosum, hippocampus and dentate gyrus, midbrain and cerebellum (FIG. 12a-h, respectively, FIG. 11). GFP-positive cells included both neurons and astrocytes throughout the brain. To further characterize the transduced neurons, brains were co-labeled for GFP and GAD67, a GABAergic marker. FIG. 13 depicts diagrams of coronal sections throughout the mouse brain corresponding to the approximate locations shown in FIG. 12a-h for postnatal day-1 injected neonatal mouse brains. The box in (13a) corresponds to the location of (FIG. 12a). The smaller box in (13b) corresponds to (FIG. 12b) and the larger box to (FIG. 12c). The larger box in (13c) corresponds to (FIG. 12d) while the smaller box in (13c) represents (FIG. 12e). Finally, (13d-f) correspond to (FIG. 12f-h) respectively.

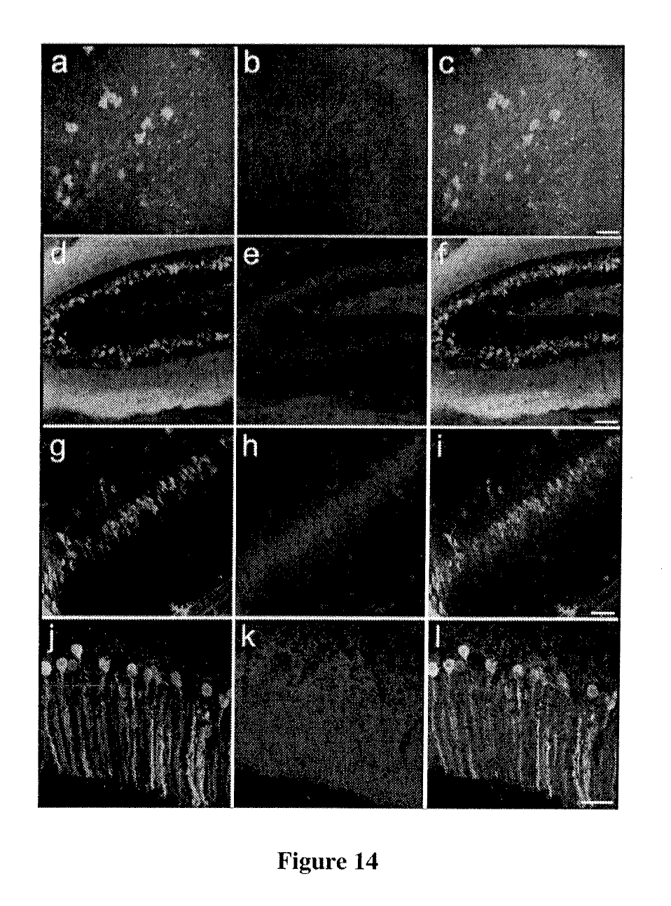

The cortex, hippocampus and dentate had very little colocalization between GFP and GAD67 labeled cells (FIG. 14a-i), while Purkinje cells in the cerebellum were extensively co-labeled (FIG. 14j-l). Finally, unbiased-estimated stereological quantification of transduction showed that 18.8+/−1.9% within the retrosplenial/cingulate cortex, 14.8+/−4.8% within the dentate gyrus and 71.8+/−3.65% within the Purkinje layer of total neurons were transduced following a one-time administration of virus (Table 1).

### Example 8

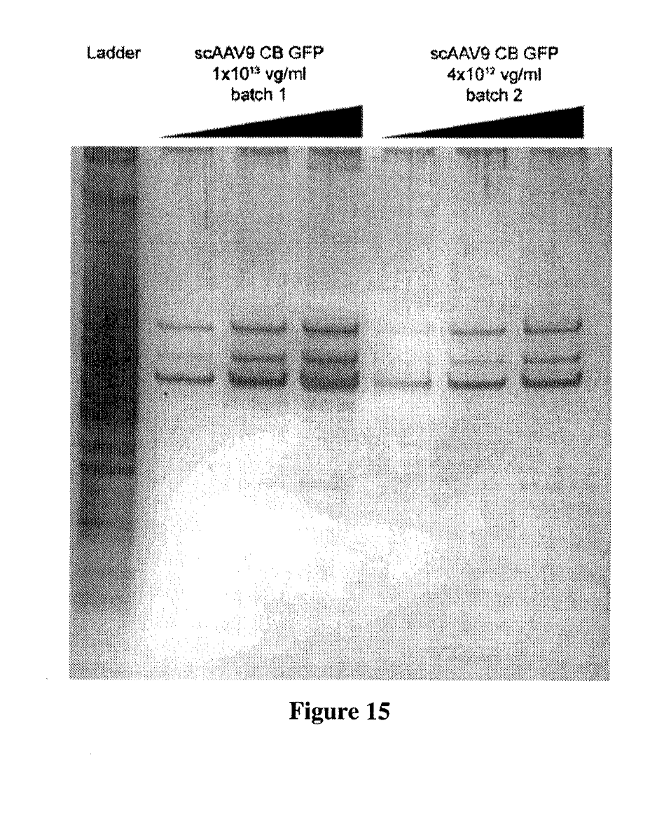

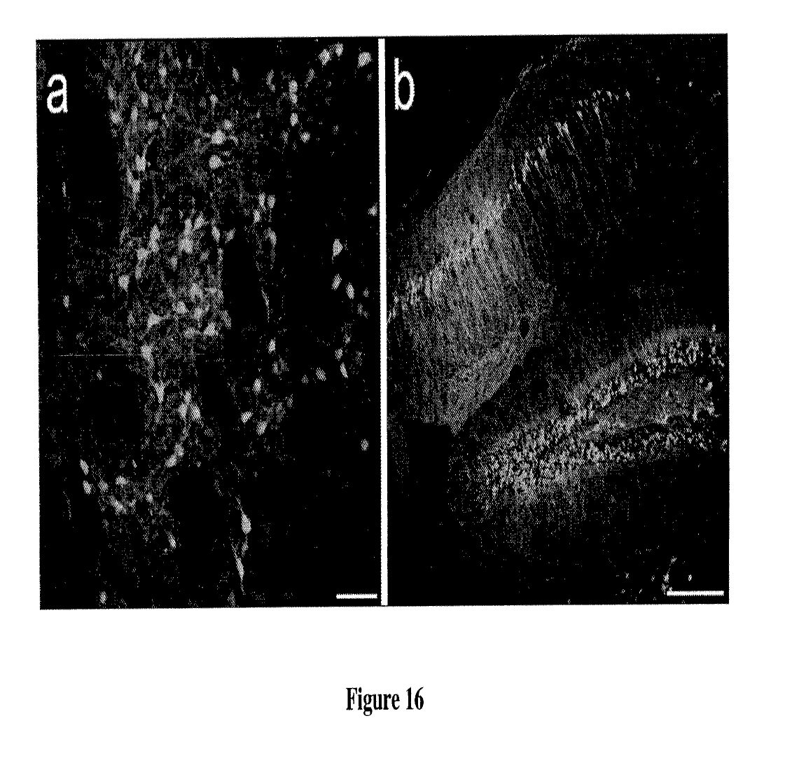

Efficient astrocyte transduction by an AAV8-, but not an AAV9-vector, following direct brain injection has been previously reported. Astrocyte transduction, however, was suggested to be related to viral purification [Klein et al., Mol Ther 16: 89-96 (2008)]. To investigate whether AAV9 astrocyte transduction was related to vector purity or delivery route, multiple AAV9 preparations were evaluated for vector purity by silver-stain and 8×10¹⁰ particles of the same scAAV9-CB-GFP vector preparations from the intravenous experiments were injected into the striatum and dentate gyrus of adult mice. Silver-staining showed that vector preparations were relatively pure and of research grade quality (FIG. 15). Two-weeks post-intracranial injection, we observed significant neuronal transduction within the injected regions using these vector preparations. However, no evidence for colocalization was found between GFP and GFAP labeling throughout the injected brains (n=3) (FIG. 16), as previously reported [Cearley et al., Mol Ther 16: 1710-1718 (2008)], suggesting the astrocyte transduction in this work may be injection route- and serotype-dependent and not due to vector purity.

The scarcity of LMN and DRG transduction seen in the adult paradigm suggests there is a developmental period in which access by circulating virus to these cell populations becomes restricted. Assuming a dependence on retrograde transport for DRG and LMN transduction following intravenous injection, Schwann cell or synapse maturation may be an important determinant of successful AAV9 LMN and DRG transduction. Direct intramuscular injection of AAV9 into adults did not result in readily detectable expression in motor neurons by retrograde transport. These results suggest that AAV9 escapes brain vasculature in a similar manner as skeletal and cardiac muscle vasculature. Once free of the vasculature, these data suggest that AAV9 infects the astrocytic-perivascular-endfeet that surround capillary endothelial cells [Abbott et al., Nat Rev Neurosci 7: 41-53 (2006)].

In summary, these results demonstrate the unique capacity of AAV9 to efficiently target cells within the CNS, and in particular widespread neuronal and motor neuron transduction in the neonate, and extensive astrocyte transduction in the adult following intravenous delivery. A simple intravenous injection of AAV9 as described herein may be clinically relevant for both SMA and ALS. In the context of SMA, data suggest that increased expression of survival motor neuron (SMN) gene in LMNs may hold therapeutic benefit [Azzouz et al., The Journal of Clinical Investigation 114: 1726-1731 (2004); Baughan et al., Mol Ther 14: 54-62 (2006)]. The importance of the results presented here is that a single injection may be able to effectively restore SMN expression levels in LMNs. Additionally, given the robust neuronal populations transduced throughout the CNS in neonatal animals, this approach may also allow for rapid, relatively inexpensive generation of chimeric animals for gene overexpression, or gene knock-down [Siegel et al., PLoS Biology 2: e419 (2004)]. Additionally, constructing AAV9 based vectors with neuronal or astrocyte specific promoters may allow further specificity, given that AAV9 targets multiple non-neuronal tissues following intravenous delivery [Inagaki et al., Mol Ther 14: 45-53 (2006); Pacak et al., Circulation Research 99: e3-9 (2006)]. The results also demonstrate efficient targeting of astrocytes in adult-treated animals, and this finding is relevant for treating ALS, where the non-cell autonomous nature of disease progression has recently been discovered, and astrocytes have been specifically linked to disease progression [Yamanaka et al., Nature Neuroscience 11: 251-253 (2008)]. The ability to target astrocytes for producing trophic factors, or to circumvent aberrant glial activity may be beneficial for treating ALS24. In sum, these data highlight a relatively non-invasive method to efficiently deliver genes to the CNS and are useful in basic and clinical neurology studies.

### Example 9

The ability of scAAV9 to traverse the blood-brain barrier in nonhuman primates [Kota et al., Sci. Transl. Med 1: 6-15 (2009)] was also investigated. A male cynomolgus macaque was intravenously injected on P1 with 1×10¹⁴ particles (2.2×10¹¹ particles/g of body weight) of scAAV9-GFP and euthanized it 25 days after injection. Examination of the spinal cord revealed robust GFP expression within the dorsal root ganglia and motor neurons along the entire neuraxis, as seen in P1-injected mice. This finding demonstrated that early systemic delivery of scAAV9 efficiently targets motor neurons in a nonhuman primate.

While the present invention has been described in terms of various embodiments and examples, it is understood that variations and improvements will occur to those skilled in the art. Therefore, only such limitations as appear in the claims should be placed on the invention.

1. A method of delivering a polynucleotide across the blood brain barrier comprising the step of systemically administering a rAAV9 comprising a self-complementary genome including the polynucleotide to a patient, wherein the polynucleotide is administered to the patient prior to completion of formation of glial cell endfeet.

2. A method of delivering a polynucleotide to the central nervous system comprising the step of systemically administering a rAAV9 comprising a self-complementary genome including the polynucleotide to a patient, wherein the polynucleotide is administered to the patient prior to completion of formation of glial cell endfeet.

3. The method of claim 1 or 2 wherein the polynucleotide is delivered to brain.

4. The method of claim 1 or 2 wherein the polynucleotide is delivered to spinal cord.

5. The method of claim 1 or 2 wherein the polynucleotide is delivered to a glial cell.

6. The method of claim 5 wherein the glial cell is an astrocyte.

7. The method of claim 1 or 2 wherein the polynucleotide is delivered to a lower motor neuron.

8. A method of delivering a polynucleotide to the peripheral nervous system comprising the step of systemically administering a rAAV9 comprising a self-complementary genome including the polynucleotide to a patient, wherein the polynucleotide is administered to the patient prior to completion of formation of glial cell endfeet.

9. The method of claim 8 wherein the polynucleotide is delivered to a nerve cell.

10. The method of claim 8 wherein the polynucleotide is delivered to a glial cell.

11. A method of treating a neurodegenerative disease comprising the step of systemically administering a rAAV9 comprising a self-complementary genome including an survival motor neuron (SMN) polynucleotide to a patient, wherein the rAAV9 is administered the patient prior to completion of formation of glial cell endfeet.

12. The method of claim 11 wherein the neurodegenerative disease is spinal muscular atrophy.

13. The method of claim 11 wherein the neurodegenerative disease is amyotrophic lateral sclerosis.

14. The method of claim 11 wherein the SMN polynucleotide is delivered to an astrocyte.

15. A method of delivering a polynucleotide to vascular endothelial cells comprising the step of systemically administering a rAAV9 comprising a self-complementary genome including the polynucleotide to a patient, wherein the polynucleotide is administered to the patient prior to completion of formation of glial cell endfeet.

16. The method of any of the preceding claims wherein the polynucleotide is administered on postnatal day 1 (P1).

17. The method of any of the preceding claims wherein the polynucleotide is administered on or before postnatal day 5 (P5).

18. The method of any of the preceding claims wherein the polynucleotide is administered on or before postnatal day 10 (P10).

19. The method of any of the preceding claims wherein the polynucleotide is administered after postnatal day 10 (P10).

20. A method of delivering a polynucleotide across endothelial cell tight junctions of the blood brain harrier comprising the step of systemically administering to a patient a rAAV9 comprising a self-complementary genome including the polynucleotide.

21. A method of delivering a polynucleotide to an astrocyte of the blood brain barrier comprising the step of systemically administering to a patient a rAAV9 comprising a self-complementary genome including the polynucleotide.

22. The method of claim 20 or 21 wherein the polynucleotide is a SMN polynucleotide.

23. The method of claim 20 or 21 wherein the polynucleotide is delivered to treat a neurodegenerative disease.

24. The method of claim 23 wherein the neurodegenerative disease is spinal muscular atrophy.

25. The method of claim 24 wherein the neurodegenerative disease is amyotrophic lateral sclerosis.

26. A rAAV9 with a self-complementary genome encoding SMN protein.

27. A rAAV with a self-complementary genome encoding a trophic or protective factor.


SEQUENCE LISTING (7 sequences)

SEQ ID NO: 1 - DNA, 1621 bp, Homo sapiens
ccacaaatgtgggagggcgataaccactcgtagaaagcgtgagaagttactacaagcggtcctcccggccaccgtactgttccgctcccagaagccccgggcggcggaagtcgtcactcttaagaagggacggggccccacgctgcgcacccgcgggtttgctatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggtttcagacaaaatcaaaaagaaggaaggtgctcacattccttaaattaaggagaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactcttttgaaaaaccatctgtaaaagactggggtgggggtgggaggccagcacggtggtgaggcagttgagaaaatttgaatgtggattagattttgaatgatattggataattattggtaattttatggcctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtatataataaaaatatttaattttttttta

SEQ ID NO: 2 - DNA, 24 bp, Artificial Sequence
  Synthetic nucleotide
tccagctccgggatattgggattg

SEQ ID NO: 3 - DNA, 23 bp, Artificial Sequence
  Synthetic nucleotide
aggtcccaccacctaagaaagcc

SEQ ID NO: 4 - DNA, 22 bp, Artificial Sequence
  Synthetic nucleotide
gtgtctgggctgtaggcattgc

SEQ ID NO: 5 - DNA, 22 bp, Artificial Sequence
  Synthetic nucleotide
gctgtgccttttggcttatctg

SEQ ID NO: 6 - DNA, 25 bp, Artificial Sequence
  Synthetic nucleotide
gcctgcgatgtcggtttctgtgagg

SEQ ID NO: 7 - DNA, 21 bp, Artificial Sequence
  Synthetic nucleotide
ccagcgcggatcggtcagacg